(19) United States
(12) Patent Application Publication
(10) Pub. No.: US 2021/0169961 A1
(43) Pub. Date: Jun. 10, 2021

(54) HEPATOPROTECTIVE TRADITIONAL CHINESE MEDICINE COMPOSITION, EXTRACT THEREOF, AND PHARMACEUTICAL USE THEREOF

(71) Applicants: SHUGUANG HOSPITAL AFFILIATED TO SHANGHAI UNIVERSITY OF TRADITIONAL CHINESE MEDICINE, Shanghai (CN); ACADEMY OF MILITARY MEDICAL SCIENCES, Beijing (CN); SHANGHAI SANG LUO MEDICINE TECHNOLOGY CO., LTD., Shanghai (CN)

(72) Inventors: Yueqiu Gao, Shanghai (CN); Junjie Shan, Beijing (CN); Xuehua Sun, Shanghai (CN); Man Li, Shanghai (CN); Yanxu Chen, Shanghai (CN); Zhenhua Zhou, Shanghai (CN); Yating Gao, Shanghai (CN); Hao Ma, Beijing (CN); Xin Zhang, Shanghai (CN); Rui Xu, Beijing (CN); Dan Nie, Beijing (CN); Lulu Liu, Beijing (CN); Changwei Li, Beijing (CN); Yu Yang, Beijing (CN); Xiunan Zhao, Beijing (CN)

(21) Appl. No.: 17/265,724

(22) PCT Filed: Dec. 18, 2018
(86) PCT No.: PCT/CN2018/121818

§ 371 Date: Feb. 3, 2021

(30) Foreign Application Priority Data
Aug. 3, 2018 (CN) 201810874325.0

(51) Int. Cl. A61K 36/746; A61K 36/074; A61K 36/481; A61K 36/71; A61K 36/489; A61K 36/537; A61K 36/296; A61K 36/64; A61K 36/284; A61K 36/752; A61K 36/41; A61K 36/488; A61K 36/72; A61K 36/53; A61K 36/9066; A61K 35/586; A61K 36/8984; A61K 36/898; A61K 36/48; A61K 36/539; A61K 9/00; A61K 9/16; A61K 9/20; A61K 9/48; A61P 31/20; A61P 1/16; A61P 35/00; A61K 31/522; A61K 31/683; A61K 31/7072; A61K 31/506; A23L 33/105; A23L 33/00

(52) U.S. Cl. CPC A61K 36/746; A23V 2002/00; A61K 36/481; A61K 36/71; A61K 36/489; A61K 36/537; A61K 36/296; A61K 36/64; A61K 36/284; A61K 36/752; A61K 36/41; A61K 36/488; A61K 36/72; A61K 36/53; A61K 36/9066; A61K 35/586; A61K 36/8984; A61K 36/898; A61K 36/48; A61K 36/539; A61K 9/0095; A61K 9/16; A61K 9/20; A61K 9/48; A61P 31/20; A61P 1/16; A61P 35/00; A61K 31/522; A61K 31/683; A61K 31/7072; A61K 31/506; A23L 33/105; A23L 33/40; A61K 2236/39; A61K 2236/53; A61K 2236/51; A61K 2236/333; A61K 36/074

(57) ABSTRACT

Disclosed is a hepatoprotective traditional Chinese medicine composition, extract and pharmaceutical use thereof. The composition comprises Morinda officinalis How, Ganoderma lucidum (Leyss. Ex Fr.) Karst., Astragalus membranaceus (Fisch) Bge. Var. monghlicus (Bge) Hsiao, Ranunculus ternatus Thunb., Sophora flavescens Alt. and Salvia miltiorrhiza Bge. The composition and extract thereof may be used for treating liver injury, hepatitis, hepatic fibrosis, hepatic cirrhosis or liver cancer, and they may also be used as a vaccine adjuvant or an immunomodulator.

### TECHNICAL FIELD

The application relates to the technical field of traditional Chinese medicines, in particular to a traditional Chinese medicine composition with the effects of resisting liver injury and modulating immunity, mainly comprising Morinda officinalis How, Ganoderma lucidum (Leyss. Ex Fr.) Karst., Astragalus membranaceus (Fisch) Bge. Var. monghlicus (Bge) Hsiao, Ranunculus ternatus Thunb., Sophora flavescens Alt. and Salvia miltiorrhiza Bge. The traditional Chinese medicine composition may be used for treating liver diseases, such as chronic hepatitis B, nonalcoholic steatohepatitis, alcoholic steatohepatitis, hepatic fibrosis, compensated cirrhosis or liver cancer (especially primary liver cancer caused by chronic hepatitis B), and it may also be used as a vaccine adjuvant or an immunomodulator.

### BACKGROUND ART

Liver is an important organ for metabolism, and it plays an important role in various physiological functions such as biosynthesis, biotransformation, detoxication, secretion, immunity and the like. However, when it is influenced by virus infection, poisons, careless medication, bad dietary habits or other pathogenic causes, the functions of liver will be impaired and result in liver injury and liver diseases, such as viral hepatitis, non-alcoholic and alcoholic liver diseases, hepatic fibrosis, hepatic cirrhosis, liver cancer, drug-induced hepatitis and so on.

In traditional Chinese medical science, liver is considered to be one of the Five Organs, and its main physiological functions are storing blood, governing catharsis, governing sinews and manifested in nails, opening at eyes and standing interior-exterior relationship with gallbladder. Liver is capable of retaining blood and regulating the amount of blood in the body. “Plain Questions—Chapter of Five Organs Generation” recites that: “thus, when a person lies down, the blood is under the control of the liver, the liver receives blood to see, the feet receive blood to walk, the hands receive blood to hold, and the fingers receive blood to grab”. Pathologically, for example, liver blood deficiency and liver blood storing failure commonly result in tiredness, fatigue, eye dryness, blurred vision, and hematemesis, hematochezia and epistaxis. The liver governs catharsis, and it can modulate Qi and blood so that Qi and blood circulate constantly, thereby ensuring normal activities of each viscera. If the liver Qi is depressed and the Qi running is not flowing, Qi stagnation and blood stasis occur; the digestive and absorptive functions of the spleen and stomach are closely related to the catharsis function of the liver. For example, if the catharsis function of the liver fails, the digestion of spleen and stomach will be influenced, resulting in the pathological change of dyspepsia. Except for symptoms of liver Qi depression, such as distending pains in chest and hypochondrium, irritability and the like, symptoms of belching, nausea, vomiting, abdominal distension and diarrhea are often caused. The liver and the gallbladder are in the exterior-interior relationship, and whether the catharsis function of liver is normal or not directly affects the secretion and excretion of the bile; if the function is normal, bile circulates along the normal path, otherwise, the bile may flow either upward or overflow and results in pathological changes, such as bitter taste, jaundice and the like. The liver stores blood and the kidney stores essence; liver blood depends on the nourishment of kidney essence, and the kidney essence will be continuously supplemented with essence transformed from liver blood; the essence and the blood are bred mutually, and so, there is the theory of “homology of liver and kidney”. Pathologically, the pathological changes of the kidney essence and liver blood often affect each other. For example, kidney essence deficiency will lead to liver blood deficiency, and in turn, the liver blood deficiency can also cause the kidney essence deficiency, resulting in symptoms, such as soreness and pain of the waist and back, spermatorrhea, tinnitus, blurred vision, dizziness, and eye dryness etc. When the liver-Yang or the liver-fire is hyperactive, the kidney-Yin will be exhausted, to result in kidney-Yin deficiency. Ascending and descending, and transportation and transformation of the spleen and stomach depend on the catharsis of liver Qi. The liver stores blood, and the spleen controls the blood and governs transportation and transformation, becoming the source of the Qi and blood. The coordination of the liver and spleen can complete the normal operation of blood. Pathologically, if Qi stagnation and blood stasis occur, the catharsis is abnormal, and the functions of the spleen and stomach in ascending and descending, and transportation and transformation are impaired, the symptoms “liver-stomach disharmony” and “liver-spleen disharmony” will occur. Frequent clinical symptoms of fullness in chest and hypochondrium, abdominal distension, ructus and vomiting, fatigue and asthenia, and abnormal bowel movement after anger are observed. Conversely, spleen diseases also affect the liver. For example, the spleen-Qi deficiency will result in deficiency of the source of blood, or the blood-control failure of the spleen and the excessive loss of blood will result in liver blood deficiency. For another example, dysfunction of the spleen in transportation and transformation will result in water and dampness retention, which are mature over time. Damp-heat stagnating causes obstruction of liver catharsis, resulting in jaundice. Clearly, liver diseases affect the spleen and in turn, the spleen diseases affect the liver, that is the liver and the spleen are pathologically interacted with each other. The heart governs blood and the liver stores blood. Thus, when blood vessels are full, there is sufficient blood which the heart can govern and the liver can store. Development of liver-Qi and descending of lung-Qi cooperate with each other to maintain normal functions of Qi movements in human bodies. To sum up, the liver and the gallbladder are connected to each other, the spleen and the stomach are communicated with each other, they connect upward to the heart and downward to the kidney. Thus, the triple energizer is unobstructed and persons will feel calm and comfortable. Hence, modern scholars who study the treatment of liver diseases focus on conditioning the spleen and the stomach, enriching water to nourish wood, smoothing Qi movement and balancing Yin and Yang, so as to realize the aim of curing liver diseases.

The traditional Chinese medical science considers that exogenous damp-heat or epidemic toxin, internal emotional distress, diet, defatigation and the like are main causes of chronic liver diseases, and the locations of the chronic liver diseases are mainly in the liver, often involving the two viscera of liver and kidney, and the hollow organ of gallbladder, stomach, and triple energizer. The natures of the diseases reside in deficient root and excessive superficial, and asthenia complicates with sthenia. Because the etiology, pathogenesis, disease locations and disease natures of chronic liver disease are complicated and changeable, and the diseases conditions are staggered and difficult to heal, the relationship between the sthenia of pathogenic factor including dampness, heat, stasis and toxin and the asthenia of healthy qi of liver, spleen and kidney should be distinguished; during treatment, the doctor should note to put person first and correctly treat the balance between strengthening the healthy qi and eliminating pathogenic factors, and the focus of the treatment shall be the adjustment of the balances in Yin-Yang, Qi-blood, and viscus functions.

Under guidance of the theory of the traditional Chinese medical science, the inventor, through long-term clinical and modern pharmacological researches, proves that kidney tonifying, spleen strengthening, dampness removing and blood circulation invigorating are basic treatment methods for treating chronic liver diseases such as chronic hepatitis B, non-alcoholic steatohepatitis, hepatic fibrosis (compensated cirrhosis), primary liver cancer caused by hepatitis B and the like. Therefore, the traditional Chinese medicine composition of the invention is provided, thereby accomplishing the present application.

Content of the Invention

Thus, in one aspect, the present application provides a traditional Chinese medicine composition, comprising the following components: Morinda officinalis How, Ganoderma lucidum (Leyss. Ex Fr.) Karst., Astragalus membranaceus (Fisch) Bge. Var. monghlicus (Bge) Hsiao, Ranunculus ternatus Thunb., Sophora flavescens Alt. and Salvia miltiorrhiza Bge. In some preferred embodiments, weight ratios of the components are: 15-35 parts of Morinda officinalis How, 10-20 parts of Ganoderma lucidum (Leyss. Ex Fr.) Karst., 25-35 parts of Astragalus membranaceus (Fisch) Bge. Var. monghlicus (Bge) Hsiao, 10-20 parts of Ranunculus ternatus Thunb., 10-20 parts of Sophora flavescens Alt., and 10-20 parts of Salvia miltiorrhiza Bge. In some preferred embodiments, weight ratios of the components are: 30 parts of Morinda officinalis How, 15 parts of Ganoderma lucidum (Leyss. Ex Fr.) Karst., 30 parts of Astragalus membranaceus (Fisch) Bge. Var. monghlicus (Bge) Hsiao, 15 parts of Ranunculus ternatus Thunb., 15 parts of Sophora flavescens Alt. and 15 parts of Salvia miltiorrhiza Bge.

In the prescription, the Morinda Officinalis How is used as a sovereign drug for tonifying the kidney-yang and kidney-Yin; the Ministerial drug is Ganoderma lucidum (Leyss. Ex Fr.) Karst. and Astragalus membranaceus (Fisch) Bge. Var. monghlicus (Bge) Hsiao for replenishing Qi to invigorate the spleen, so as to achieve that “the liver and the kidney are simultaneously tonified and the congenital and postnatal deficiencies are simultaneously conditioned”, and simultaneously tonify the kidney and the spleen; Sophora flavescens Alt. and Ranunculus ternatus Thunb. are used as adjuvants for clearing heat, removing dampness and detoxifying, thereby “eliminating remaining pathogenic factors”; Salvia miltiorrhiza Bge. is used to promote Qi and activate blood circulation, and to guide drugs into meridian. The whole prescription is used together, wherein tonifying kidney and spleen is the primary aspect of disease treatment while clearing heat and removing dampness are the secondary aspect of disease treatment; both the primary and the secondary symptoms should be relieved whilst promoting Qi and activating blood circulation, thereby strengthening organs and restoring vitality, clearing dampness, relieving all symptoms, and the disease is cured.

In some preferred embodiments, the traditional Chinese medicine composition further comprises one or more (e.g., 2-10, e.g., 2-6, e.g., 2, 3, 4, 5, or 6) of the following components: Epimedium brevicornu Maxim., Rehmannia glutinosa Libosch., Atractylodes macrocephala Koidz., Citrus reticulata Blanco, Sedum sarmentosum Bunge, Pueraria lobata (Willd.) Ohwi, Hovenia acerba Lindl., Flower of Pueraria lobata (Willd.) Ohwi, Lycopus lucidus Turcz. var. hirtus Regel, Curcuma wenyuj in Y. H. Chen et C. Ling, Stir-baked Trionyx sinensis Wiegmann, Dendrobium nobile Lindl., Cremastra appendiculata Makino, Abrus cantoniensis Hance, Curcuma phaeocaulis Val., and Scutellaria barbata D. Don.

In some preferred embodiments, the traditional Chinese medicine composition further comprises the following components: Epimedium brevicornu Maxim., Rehmannia glutinosa Libosch., Atractylodes macrocephala Koidz., and Citrus reticulata Blanco. In some preferred embodiments, the weight ratios of the components are: 15-35 parts of Morinda officinalis How, 10-20 parts of Ganoderma lucidum (Leyss. Ex Fr.) Karst., 25-35 parts of Astragalus membranaceus (Fisch) Bge. Var. monghlicus (Bge) Hsiao, 10-20 parts of Ranunculus ternatus Thunb., 10-20 parts of Sophora flavescens Alt., 10-20 parts of Salvia miltiorrhiza Bge., 10-30 parts of Epimedium brevicornu Maxim., 5-15 parts of Rehmannia glutinosa Libosch., 10-30 parts of Atractylodes macrocephala Koidz., and 5-10 parts of Citrus reticulata Blanco. In some preferred embodiments, the weight ratios of the components are: 30 parts of Morinda officinalis How, 15 parts of Ganoderma lucidum (Leyss. Ex Fr.) Karst., 30 parts of Astragalus membranaceus (Fisch) Bge. Var. monghlicus (Bge) Hsiao, 15 parts of Ranunculus ternatus Thunb., 15 parts of Sophora flavescens Alt., 15 parts of Salvia miltiorrhiza Bge., 15 parts of Epimedium brevicornu Maxim., 10 parts of Rehmannia glutinosa Libosch., 15-30 parts of Atractylodes macrocephala Koidz. and 9-10 parts of Citrus reticulata Blanco.

Pharmacological researches prove that the above prescription can significantly improve symptoms, like liver enlargement, of ConA- and LPS-induced Balb/C liver injury model mice, inhibit hepatocyte necrosis and inflammatory cells aggregation, and increase division and proliferation of hepatocytes; it can also significantly promote proliferations of hepatocytes and also effectively inhibit secretions of hepatitis B virus antigens HBsAg and HBeAg; moreover, it has evident immune-modulating effects, and for example, it has remarkable proliferation promoting activity on mouse splenocytes, to promote T cells and B cells proliferation and promote mouse splenocytes to secrete IFN-γ and TNF-α. In clinical tests, the prescription can significantly improve common symptoms of patients with HBeAg negative chronic hepatitis B and remarkably increase recurrence rate of serum ALT and AST. The prescription can be used for treating liver diseases (such as chronic hepatitis B) and liver injury (especially drug- or toxicant-induced liver injury), and it can be used as an immunological adjuvant or an immunomodulator.

In some preferred embodiments, the traditional Chinese medicine composition further comprises the following components: Sedum sarmentosum Bunge and Pueraria lobata (Willd.) Ohwi. In some preferred embodiments, the weight ratios of the components are: 15-35 parts of Morinda officinalis How, 10-20 parts of Ganoderma lucidum (Leyss. Ex Fr.) Karst., 25-35 parts of Astragalus membranaceus (Fisch) Bge. Var. monghlicus (Bge) Hsiao, 10-20 parts of Ranunculus ternatus Thunb., 10-20 parts of Sophora flavescens Alt., 10-20 parts of Salvia miltiorrhiza Bge., 15-30 parts of Sedum sarmentosum Bunge, and 15-30 parts of Pueraria lobata (Willd.) Ohwi. In some preferred embodiments, the weight ratios of the components are: 30 parts of Morinda officinalis How, 15 parts of Ganoderma lucidum (Leyss. Ex Fr.) Karst., 30 parts of Astragalus membranaceus (Fisch) Bge. Var. monghlicus (Bge) Hsiao, 15 parts of Ranunculus ternatus Thunb., 10 parts of Sophora flavescens Alt., 15 parts of Salvia miltiorrhiza Bge., 30 parts of Sedum sarmentosum Bunge and 30 parts of Pueraria lobata (Willd.) Ohwi.

Pharmacological researches show that the above prescription can promote hepatocyte proliferation and have evident immune-modulating effect. For example, it has remarkable proliferation promoting activity on mouse splenocytes, and promote T cell and B cell proliferations. In clinical tests, the prescription can remarkably improve the liver functions of a patient with nonalcoholic steatohepatitis, reduce the blood fat level and increase the liver/spleen CT value grades. The prescription can be used for treating liver diseases (such as nonalcoholic steatohepatitis) and liver injury (especially drug- or toxicant-induced liver injury), and it can be used as an immunological adjuvant or an immunomodulator.

In some preferred embodiments, the traditional Chinese medicine composition further comprises the following components: following components: Sedum sarmentosum Bunge, Pueraria lobata (Willd.) Ohwi, Hovenia acerba Lindl. and Flower of Pueraria lobata (Willd.) Ohwi. In some preferred embodiments, the weight ratios of the components are: 15-35 parts of Morinda officinalis How, 10-20 parts of Ganoderma lucidum (Leyss. Ex Fr.) Karst., 25-35 parts of Astragalus membranaceus (Fisch) Bge. Var. monghlicus (Bge) Hsiao, 10-20 parts of Ranunculus ternatus Thunb., 10-20 parts of Sophora flavescens Alt., 10-20 parts of Salvia miltiorrhiza Bge., 15-30 parts of Sedum sarmentosum Bunge, 15-30 parts of Pueraria lobata (Willd.) Ohwi, 15-30 parts of Hovenia acerba Lindl., and 10-20 parts of Flower of Pueraria lobata (Willd.) Ohwi. In some preferred embodiments, the weight ratios of the components are: 30 parts of Morinda officinalis How, 15 parts of Ganoderma lucidum (Leyss. Ex Fr.) Karst., 30 parts of Astragalus membranaceus (Fisch) Bge. Var. monghlicus (Bge) Hsiao, 15 parts of Ranunculus ternatus Thunb., 10 parts of Sophora flavescens Alt. 15 parts of Salvia miltiorrhiza Bge., 30 parts of Sedum sarmentosum Bunge, 30 parts of Pueraria lobata (Willd.) Ohwi, 30 parts of Hovenia acerba Lindl., and 15 parts of Flower of Pueraria lobata (Willd.) Ohwi.

Pharmacological researches show that the above prescription can promote hepatocyte proliferations and have evident immune-modulating effects. For example, it has remarkable proliferation promoting activity on mouse splenocytes, and promote T cell and B cell proliferations. In clinical tests, the prescription can remarkably reduce levels of ALT, AST, GGT and total cholesterol of a patient with alcoholic steatohepatitis, and evidently improve symptoms in traditional Chinese medicine, like lassitude and anorexia. The prescription can be used for treating liver diseases (such as alcoholic steatohepatitis) and liver injury (especially drug- or toxicant-induced liver injury), and it can be used as an immunological adjuvant or an immunomodulator.

In some preferred embodiments, the traditional Chinese medicine composition further comprises the following components: Lycopus lucidus Turcz. var. hirtus Regel, Curcuma wenyujin Y. H. Chen et C. Ling, Stir-baked Trionyx sinensis Wiegmann and Dendrobium nobile Lindl. In some preferred embodiments, the weight ratios of the components are: 15-35 parts of Morinda officinalis How, 10-20 parts of Ganoderma lucidum (Leyss. Ex Fr.) Karst., 25-35 parts of Astragalus membranaceus (Fisch) Bge. Var. monghlicus (Bge) Hsiao, 10-20 parts of Ranunculus ternatus Thunb., 10-20 parts of Sophora flavescens Alt., 10-20 parts of Salvia miltiorrhiza Bge., 10-20 parts of Lycopus lucidus Turcz. var. hirtus Regel, 10-20 parts of Curcuma wenyujin Y. H. Chen et C. Ling, 10-20 parts of Stir-baked Trionyx sinensis Wiegmann and 10-20 parts of Dendrobium nobile Lindl. In some preferred embodiments, the weight ratios of the components are: 30 parts of Morinda officinalis How, 15 parts of Ganoderma lucidum (Leyss. Ex Fr.) Karst., 30 parts of Astragalus membranaceus (Fisch) Bge. Var. monghlicus (Bge) Hsiao, 15 parts of Ranunculus ternatus Thunb., 10 parts of Sophora flavescens Alt., 15 parts of Salvia miltiorrhiza Bge., 15 parts of Lycopus lucidus Turcz. var. hirtus Regel, 15 parts of Curcuma wenyujin Y. H. Chen et C. Ling, 15 parts of Stir-baked Trionyx sinensis Wiegmann and 15 parts of Dendrobium nobile Lindl.

Pharmacological researches show that the above prescription can promote hepatocyte proliferations and have evident immune-modulating effects. For example, it has remarkable proliferation promoting activity on mouse splenocytes, to promote T cell and B cell proliferations. In clinical tests, the prescription can remarkably reduce the HBeAg level of a patient with hepatic fibrosis and improve fat inflammation grades. Moreover, the prescription can remarkably reduce CTP scores of patients in the compensated hepatitis B cirrhosis and increase CLDQ scores. The prescription can be used for treating liver diseases (such as hepatic fibrosis and compensated cirrhosis) and liver injury (especially drug- or toxicant-induced liver injury), and it can be used as an immunological adjuvant or an immunomodulator.

In some preferred embodiments, the traditional Chinese medicine composition further comprises the following components: Cremastra appendiculata Makino, Abrus cantoniensis Hance, Curcuma phaeocaulis Val., Atractylodes macrocephala Koidz. and Scutellaria barbata D. Don. In some preferred embodiments, the weight ratios of the components are: 15-35 parts of Morinda officinalis How, 10-20 parts of Ganoderma lucidum (Leyss. Ex Fr.) Karst., 25-35 parts of Astragalus membranaceus (Fisch) Bge. Var. monghlicus (Bge) Hsiao, 10-20 parts of Ranunculus ternatus Thunb., 10-20 parts of Sophora flavescens Alt., 10-20 parts of Salvia miltiorrhiza Bge., 5-10 parts of Cremastra appendiculata Makino, 15-30 parts of Abrus cantoniensis Hance, 5-10 parts of Curcuma phaeocaulis Val., 20-30 parts of Atractylodes macrocephala Koidz. and 10-20 parts of Scutellaria barbata D. Don. In some preferred embodiments, the weight ratios of the components are: 30 parts of Morinda officinalis How, 15 parts of Ganoderma lucidum (Leyss. Ex Fr.) Karst., 30 parts of Astragalus membranaceus (Fisch) Bge. Var. monghlicus (Bge) Hsiao, 15 parts of Ranunculus ternatus Thunb., 10 parts of Sophora flavescens Alt., 15 parts of Salvia miltiorrhiza Bge., 6 parts of Cremastra appendiculata Makino, 15 parts of Abrus cantoniensis Hance, 6 parts of Curcuma phaeocaulis Val., 15 parts of Atractylodes macrocephala Koidz. and 15 parts of Scutellaria barbata D. Don.

Pharmacological researches show that the above prescription can promote hepatocyte proliferations and have evident immune-modulating effects. For example, it has remarkable proliferation promoting activity on mouse splenocytes, to promote T cell and B cell proliferations. In clinical tests, the prescription can remarkably prolong the life span of patients with primary liver cancer, reduce recurrence rates and improve main traditional Chinese medical symptoms of patients with primary liver cancer after surgery. The prescription can be used for treating liver diseases (such as liver cancer, especially primary liver cancer caused by chronic hepatitis B) and liver injury (especially drug- or toxicant-induced liver injury), and it can be used as an immunological adjuvant or an immunomodulator.

In another aspect, the present application provides a traditional Chinese medicine extract A, which is prepared through the following method:

mixing the abovementioned components of the traditional Chinese medicine composition, adding water and decocting to give a water decoction liquor;

concentrating the water decoction liquor to give a concentrated liquor, namely the traditional Chinese medicine extract A.

In some preferred embodiments, the volume of the water added is 5 to 20 times (in L: Kg) (e.g., 5, 10, 15, or 20 times) relative to the weight of the components.

In some preferred embodiments, prior to adding water and decocting, the method further comprises a step of comminuting the components.

In some preferred embodiments, after the comminuting, the method further comprises a step of screening through a sieve (e.g., a 20-mesh sieve).

In some preferred embodiments, prior to concentrating, the method further comprises steps of filtering and/or centrifuging the water decoction liquor.

In some preferred embodiments, the method further comprises a step of drying (e.g., freeze drying) the concentrated liquor.

In another aspect, the present application provides a traditional Chinese medicine extract B, which is prepared through the following method:

adding ethanol to the abovementioned concentrated liquor and letting it on stand for 12-72 h (such as 12 h, 24 h, 36 h, 48 h or 72 h) for precipitation;

collecting a supernatant and concentrating it to provide the traditional Chinese medicine extract B.

In some preferred embodiments, the volume of the ethanol added is 1 to 10 times (in L: Kg) (e.g., 2, 3, 5, 8, or 10 times) relative to the volume of the concentrated liquor.

In some preferred embodiments, after the precipitation and prior to collecting the supernatant, the method further comprises a step of centrifuging.

In some preferred embodiments, after the concentrating, the method further comprises a step of drying.

In another aspect, the present application provides a traditional Chinese medicine extract C, which is prepared through the following method:

adding ethanol to the abovementioned concentrated liquor and letting it on stand for 12-72 h (such as 12 h, 24 h, 36 h, 48 h or 72 h) for precipitation;

collecting the precipitate and dissolving it in water in a manner of low intensity and high frequency, intercepting the fraction with molecular weight >1000 Da by dialysis, and concentrating to provide the traditional Chinese medicine extract C.

In some preferred embodiments, the volume of the ethanol added is 1 to 10 times (e.g., 2, 3, 5, 8, or 10 times) relative to the volume of the concentrated liquor.

In some preferred embodiments, the total volume of the water added is 1-3 times (in L: kg) relative to the weight of the medicine materials.

In some preferred embodiments, after the concentrating, the method further comprises a step of drying.

In another aspect, the present application provides a pharmaceutical composition, comprising the abovementioned traditional Chinese medicine composition or the traditional Chinese medicine extract, and optionally one or more pharmaceutically acceptable carriers or excipients.

The pharmaceutically acceptable carrier refers to a diluent, an adjuvant, an excipient, or a vehicle together with which therapeutic agents are administered, and within the scope of reasonable medical judgments, it is suitable to contact with tissues of human being and/or other animals without excessive toxicities, irritations, nor provoking allergic responses, or other problems or complications commensurate with reasonable benefit/risk ratios.

Pharmaceutically acceptable carriers that can be used in the pharmaceutical compositions of the present application include, but are not limited to, sterile liquids, such as water and oil, including those oils sourced from petroleum, animal, vegetable or man-made oil, such as peanut oil, soybean oil, mineral oil, sesame oil and the like.

When the pharmaceutical composition is administered intravenously, water is an exemplary carrier. Physiological saline, aqueous solution of dextrose and glycerol can also be used as a liquid carrier, particularly for injectable solutions. Suitable pharmaceutical excipients include starch, glucose, lactose, sucrose, gelatin, maltose, chalk, silica gel, sodium stearate, glycerol monostearate, talc, sodium chloride, defatted milk powder, glycerol, propylene glycol, water, ethanol and the like. The pharmaceutical composition, if necessary, may comprise a small quantity of a wetting agent, an emulsifier or a pH buffering agent.

An oral formulation may comprise a standard carrier, such as, in a pharmaceutical grade, mannitol, lactose, starch, magnesium stearate, sodium saccharine, cellulose, magnesium carbonate, and the like. Examples of suitable pharmaceutically acceptable carriers are described in Remington's Pharmaceutical Sciences (1990).

The pharmaceutical compositions of the present application may systemically and/or locally act. For this purpose, they may be administered by a suitable route, for example in an injection, intravenous, intraarterial, subcutaneous, intraperitoneal, intramuscular or transdermal form; or they may be administrated in oral, buccal, nasal, transmucosal, and topical forms, in a form of ophthalmic preparations or by inhalation.

For these administration routes, the pharmaceutical compositions of the present application may be administered in suitable dosage forms. Such dosage forms include, but are not limited to, tablets, capsules, lozenges, hard candies, decoctions, oral liquids, powders, medical teas, medical wines, granules, pills, extracts, sprays, creams, ointments, suppositories, gels, pastes, lotions, ointments, aqueous suspensions, injectable solutions, elixirs, syrups. In some preferred embodiments, the pharmaceutical compositions may be formulated into decoctions, oral liquids, medical teas, medical wines, granules, tablets, capsules, powder, pills, or extracts.

In another aspect, the present application provides a pharmaceutical combination, comprising the abovementioned traditional Chinese composition, the traditional Chinese medicine extract or the pharmaceutical composition, and one or more antiviral drugs.

In some preferred embodiments, the antiviral drug is a nucleoside antiviral drug. In some preferred embodiments, the antiviral drug is selected from the group consisting of Entecavir, Tenofovir, Telbivudine, Adefovir, and Lamivudine, preferably Entecavir.

In some preferred embodiments, the traditional Chinese medicine composition, traditional Chinese medicine extracts or pharmaceutical composition and the antiviral drugs are in the same formulation unit. In some preferred embodiments, the traditional Chinese medicine composition, traditional Chinese medicine extracts or pharmaceutical composition and the antiviral drugs are in different formulation units. In some preferred embodiments, the traditional Chinese medicine, traditional Chinese medicine extracts or pharmaceutical composition and the antiviral drugs are administered simultaneously, separately or sequentially.

In another aspect, the present application provides an adjuvant or an immunomodulator, comprising the previously described traditional Chinese medicine composition, traditional Chinese medicine extracts or pharmaceutical composition.

The term “adjuvant” refers to a substance capable of non-specifically enhancing immune responses to antigens, which is injected into organisms prior to or together with the antigens, and it can enhance immune responses of the organisms to the antigen or change the type of immune responses.

The term “immunomodulator” refers to a formulation capable of modulating, balancing and recovering immune functions of organisms. The commonly used immunomodulator includes an immune promoter, an immune inhibitor and an immune bidirectional modulator.

In another aspect, the present application provides use of the previously described traditional Chinese medicine composition, traditional Chinese medicine extracts or pharmaceutical composition or pharmaceutical combination in the manufacture of a medicament for the treatment of liver injury or a liver disease.

In another aspect, the present application provides the previously described traditional Chinese medicine composition, traditional Chinese medicine extracts or pharmaceutical composition or pharmaceutical combination, for use in the treatment of liver injury or a liver disease.

In another aspect, the present application provides a method for treating liver injury or a liver disease, comprising the step of administering an effective amount of the previously described traditional Chinese medicine composition, the traditional Chinese medicine extract, the pharmaceutical composition or the pharmaceutical combination to an individual in need of such treatment.

The term “effective amount” refers to an amount of a compound that, after being administrated, will relieve one or more symptoms of disease to be treated to an extent. The dose of the traditional Chinese medicine composition, traditional Chinese medicine extracts or pharmaceutical composition described herein primarily depends on individuals to be treated, disorder or condition severities, administration rates and judgments of prescribers. Generally, the effective dose is from 130 to 200 g drug/day per kg body weight. In some cases, a dose level that is not higher than the lower limit of the previously described range may be enough. However, in other cases, a high dose may be utilized in the case of not causing any harmful side effects, provided that the high dose is first divided into several small doses for administrations throughout the day.

The term “treating” refers to blocking, relieving, or inhibiting progresses of diseases or conditions or one or more symptoms of the diseases or conditions, or preventing one or more symptoms of the diseases or conditions.

The term “individual” includes humans or non-human animals. Exemplary human individuals include individuals (referred to as patients) suffered from diseases (e.g., the diseases described herein) or normal individuals. The non-human animals include all vertebrates, for example non-mammals (such as birds, amphibians, and reptiles) and mammals, for example non-human primates, livestock and/or domestic animals (such as sheep, dogs, cats, cows, pigs, etc.).

In some preferred embodiments, the liver disease is selected from chronic hepatitis (e.g. chronic hepatitis B), nonalcoholic steatohepatitis, alcoholic steatohepatitis, hepatic fibrosis, compensated cirrhosis, liver cancer (especially primary liver cancer caused by chronic hepatitis B).

In some preferred embodiments, the liver injury is the liver injury caused by drug or toxicant.

Also disclosed is use of the previously described traditional Chinese medicine composition, the traditional Chinese medicine extract, or the pharmaceutical composition as an adjuvant or immunomodulator.

### Advantageous Effect of the Invention

The present application provides a traditional Chinese medicine composition having the activity of resisting liver injury and modulating immunity, mainly comprising Morinda officinalis How, Ganoderma lucidum (Leyss. Ex Fr.) Karst., Astragalus membranaceus (Fisch) Bge. Var. monghlicus (Bge) Hsiao, Ranunculus ternatus Thunb., Sophora flavescens Alt. and Salvia miltiorrhiza Bge. The traditional Chinese medicine composition is effective in treating liver diseases, resisting liver injury and modulating immunity. Pharmacological studies show that, in some preferred embodiments, the traditional Chinese medicine composition of the present application has evident effects of resisting inflammatory injuries and oxidative injuries of the liver, promoting hepatocyte regeneration, inhibiting the secretion of hepatocyte HBsAg and HBeAg, and inhibiting replications of hepatitis B virus to some extent. It can be used for treating liver diseases, for example chronic hepatitis (such as chronic hepatitis B), nonalcoholic steatohepatitis, alcoholic steatohepatitis, hepatic fibrosis, compensated cirrhosis or liver cancer (especially primary liver cancer caused by chronic hepatitis B), for resisting liver injury (especially drug- or toxicant-induced liver injury), and also as an immunomodulator or a vaccine adjuvant.

### DESCRIPTION OF THE DRAWINGS

FIG. 1 shows the effect of the compound dry extract prepared in Example 21 on ConA-induced liver injury in mice through pathology analysis.

### SPECIFIC MODE FOR CARRYING OUT THE INVENTION

Embodiments of the present invention will be described in detail below with reference to examples. However, a person skilled in the art shall understand that the following examples are only illustrative for the invention but should not be deemed as restricting the scope of the invention. The examples in which specific conditions are not specified, are carried out according to conventional conditions or conditions recommended by the manufacturers. The reagents or instruments used without indicating manufacturers are all conventional products that are commercially available.

### Example 1: Preparation of the Decoction

The medicine materials were weighed according to the following weights: 30 g of Morinda officinalis How, 15 g of Ganoderma lucidum (Leyss. Ex Fr.) Karst., 30 g of Astragalus membranaceus (Fisch) Bge. Var. monghlicus (Bge) Hsiao, 15 g of Sophora flavescens Alt., 15 g of Ranunculus ternatus Thunb., 15 g of Salvia miltiorrhiza Bge., 15 g of Epimedium brevicornu Maxim., 10 g of Rehmannia glutinosa Libosch., 15 g of Atractylodes macrocephala Koidz. and 10 g of Citrus reticulata Blanco. After being mixed, the medicine materials were decocted with water for 2 times, and the volume of the water added for each time was 15 times relative to the weight of the medicine materials (V/W, L/kg, the same below). The decoction liquors were combined to prepare the decoction.

### Example 2 Preparation of the Decoction

The medicine materials were weighed according to the following weights: 30 g of Morinda officinalis How, 15 g of Ganoderma lucidum (Leyss. Ex Fr.) Karst., 30 g of Astragalus membranaceus (Fisch) Bge. Var. monghlicus (Bge) Hsiao, 10 g of Sophora flavescens Alt., 15 g of Ranunculus ternatus Thunb., 15 g of Salvia miltiorrhiza Bge., 30 g of Sedum sarmentosum Bunge and 30 g of Pueraria lobata (Willd.) Ohwi. After being mixed, the medicine materials were decocted with water for 2 times, and the volume of the water added for each time was 10 times relative to the weight of the medicine materials (V/W, L/kg). The decoction liquors were combined to prepare the decoction.

### Example 3 Preparation of the Decoction

The medicine materials were weighed according to the following weights: 30 g of Morinda officinalis How, 15 g of Ganoderma lucidum (Leyss. Ex Fr.) Karst., 30 g of Astragalus membranaceus (Fisch) Bge. Var. monghlicus (Bge) Hsiao, 10 g of Sophora flavescens Alt., 15 g of Ranunculus ternatus Thunb., 15 g of Salvia miltiorrhiza Bge., 30 g of Sedum sarmentosum Bunge, 30 g of Pueraria lobata (Willd.) Ohwi, 30 g of Hovenia acerba Lindl., and 15 g of Flower of Pueraria lobata (Willd.) Ohwi. After being mixed, the medicine materials were decocted with water for 2 times, and the volume of the water added for each time was 10 times relative to the weight of the medicine materials (V/W, L/kg). The decoction liquors were combined to prepare the decoction.

### Example 4 Preparation of the Decoction

The medicine materials were weighed according to the following weights: 30 g of Morinda officinalis How, 15 g of Ganoderma lucidum (Leyss. Ex Fr.) Karst., 30 g of Astragalus membranaceus (Fisch) Bge. Var. monghlicus (Bge) Hsiao, 10 g of Sophora flavescens Alt., 15 g of Ranunculus ternatus Thunb., 15 g of Salvia miltiorrhiza Bge., 15 g of Lycopus lucidus Turcz. var. hirtus Regel, 15 g of Curcuma wenyujin Y. H. Chen et C. Ling, 15 g of Stir-baked Trionyx sinensis Wiegmann, and 15 g of Dendrobium nobile Lindl. After being mixed, the medicine materials were decocted with water for 2 times, and the volume of the water added for each time was 10 times relative to the weight of the medicine materials (V/W, L/kg). The decoction liquors were combined to prepare the decoction.

### Example 5 Preparation of the Decoction

The medicine materials were weighed according to the following weights: 30 g of Morinda officinalis How, 15 g of Ganoderma lucidum (Leyss. Ex Fr.) Karst., 30 g of Astragalus membranaceus (Fisch) Bge. Var. monghlicus (Bge) Hsiao, 10 g of Sophora flavescens Alt., 15 g of Ranunculus ternatus Thunb., 15 g of Salvia miltiorrhiza Bge., 6 g of Cremastra appendiculata Makino, 15 g of Abrus cantoniensis Hance, 6 g of Curcuma phaeocaulis Val., 15 g of Atractylodes macrocephala Koidz., 15 g of Scutellaria barbata D. Don. After being mixed, the medicine materials were decocted with water for 2 times, and the volume of the water added for each time was 10 times relative to the weight of the medicine materials (V/W, L/kg). The decoction liquors were combined to prepare the decoction.

### Example 6: Preparation of Granules

The medicine materials were weighed according to the following weights: 30 g of Morinda officinalis How, 15 g of Ganoderma lucidum (Leyss. Ex Fr.) Karst., 30 g of Astragalus membranaceus (Fisch) Bge. Var. monghlicus (Bge) Hsiao, 15 g of Sophora flavescens Alt., 15 g of Ranunculus ternatus Thunb., 15 g of Salvia miltiorrhiza Bge., 15 g of Epimedium brevicornu Maxim., 10 g of Rehmannia glutinosa Libosch., 15 g of Atractylodes macrocephala Koidz. and 10 g of Citrus reticulata Blanco. The medicine materials were mixed and comminuted, then screened by a 20-mesh sieve, followed by decoction with water for 2 times, and the volume of the water added for each time was 15 times relative to the weight of the medicine materials (V/W, L/kg). The decoction liquor was filtrated, the filtrate was centrifuged, and the supernatant was concentrated to provide a clear extract. The clear extract was added with sucrose and other adjuvants, then pelletized and dried to produce the granules.

### Example 7: Preparation of Granules

The medicine materials were weighed according to the following weights: 30 g of Morinda officinalis How, 15 g of Ganoderma lucidum (Leyss. Ex Fr.) Karst., 30 g of Astragalus membranaceus (Fisch) Bge. Var. monghlicus (Bge) Hsiao, 10 g of Sophora flavescens Alt., 15 g of Ranunculus ternatus Thunb., 15 g of Salvia miltiorrhiza Bge., 30 g of Sedum sarmentosum Bunge and 30 g of Pueraria lobata (Willd.) Ohwi. The medicine materials were mixed and comminuted, then screened by a 20-mesh sieve, followed by decoction with water for 2 times, and the volume of the water added for each time was 15 times relative to the weight of the medicine materials (V/W, L/kg). The decoction liquor was filtrated, the filtrate was centrifuged, and the supernatant was concentrated to provide a clear extract. The clear extract was added with sucrose and other adjuvants, then pelletized and dried to produce the granules.

### Example 8: Preparation of Granules

The medicine materials were weighed according to the following weights: 30 g of Morinda officinalis How, 15 g of Ganoderma lucidum (Leyss. Ex Fr.) Karst., 30 g of Astragalus membranaceus (Fisch) Bge. Var. monghlicus (Bge) Hsiao, 10 g of Sophora flavescens Alt., 15 g of Ranunculus ternatus Thunb., 15 g of Salvia miltiorrhiza Bge., 30 g of Sedum sarmentosum Bunge, 30 g of Pueraria lobata (Willd.) Ohwi, 30 g of Hovenia acerba Lindl., and 15 g of Flower of Pueraria lobata (Willd.) Ohwi. The medicine materials were mixed and comminuted, then screened by a 20-mesh sieve, followed by decoction with water for 2 times, and the volume of the water added for each time was 15 times relative to the weight of the medicine materials (V/W, L/kg). The decoction liquor was filtrated, the filtrate was centrifuged, and the supernatant was concentrated to provide a clear extract. The clear extract was added with sucrose and other adjuvants, then pelletized and dried to produce the granules.

### Example 9: Preparation of Granules

The medicine materials were weighed according to the following weights: 30 g of Morinda officinalis How, 15 g of Ganoderma lucidum (Leyss. Ex Fr.) Karst., 30 g of Astragalus membranaceus (Fisch) Bge. Var. monghlicus (Bge) Hsiao, 10 g of Sophora flavescens Alt., 15 g of Ranunculus ternatus Thunb., 15 g of Salvia miltiorrhiza Bge., 15 g of Lycopus lucidus Turcz. var. hirtus Regel, 15 g of Curcuma wenyujin Y. H. Chen et C. Ling, 15 g of Stir-baked Trionyx sinensis Wiegmann, and 15 g of Dendrobium nobile Lindl. The medicine materials were mixed and comminuted, then screened by a 20-mesh sieve, followed by decoction with water for 2 times, and the volume of the water added for each time was 15 times relative to the weight of the medicine materials (V/W, L/kg). The decoction liquor was filtrated, the filtrate was centrifuged, and the supernatant was concentrated to provide a clear extract. The clear extract was added with sucrose and other adjuvants, then pelletized and dried to produce the granules.

### Example 10: Preparation of Granules

The medicine materials were weighed according to the following weights: 30 g of Morinda officinalis How, 15 g of Ganoderma lucidum (Leyss. Ex Fr.) Karst., 30 g of Astragalus membranaceus (Fisch) Bge. Var. monghlicus (Bge) Hsiao, 10 g of Sophora flavescens Alt., 15 g of Ranunculus ternatus Thunb., 15 g of Salvia miltiorrhiza Bge., 6 g of Cremastra appendiculata Makino, 15 g of Abrus cantoniensis Hance, 6 g of Curcuma phaeocaulis Val., 15 g of Atractylodes macrocephala Koidz., 15 g of Scutellaria barbata D. Don. The medicine materials were mixed and comminuted, then screened by a 20-mesh sieve, followed by decoction with water for 2 times, and the volume of the water added for each time was 15 times relative to the weight of the medicine materials (V/W, L/kg). The decoction liquor was filtrated, the filtrate was centrifuged (3000 r/min), and the supernatant was concentrated to provide a clear extract. The clear extract was added with sucrose and other adjuvants, then pelletized and dried to produce the granules.

### Example 11: Preparation of Tablets

The medicine materials were weighed according to the following weights: 30 g of Morinda officinalis How, 15 g of Ganoderma lucidum (Leyss. Ex Fr.) Karst., 30 g of Astragalus membranaceus (Fisch) Bge. Var. monghlicus (Bge) Hsiao, 15 g of Sophora flavescens Alt., 15 g of Ranunculus ternatus Thunb., 15 g of Salvia miltiorrhiza Bge., 15 g of Epimedium brevicornu Maxim., 10 g of Rehmannia glutinosa Libosch., 15 g of Atractylodes macrocephala Koidz. and 10 g of Citrus reticulata Blanco. The medicine materials were decocted with water for 2 times, and the volume of the water added for each time was 15 times relative to the weight of the medicine materials (V/W, L/kg), and decocted for 1.5 h. The decoction liquors were combined and filtrated, the filtrate was concentrated to give a dense extract which was further added with suitable amounts of microcrystalline cellulose, croscarmellose sodium and magnesium stearate and mixed uniformly. 5% PVPK30 solution was added with stirring to produce a soft material which was screened through a 16-mesh sieve for granulation, then tableted and coated to produce the tablets.

### Example 12: Preparation of Tablets

The medicine materials were weighed according to the following weights: 30 g of Morinda officinalis How, 15 g of Ganoderma lucidum (Leyss. Ex Fr.) Karst., 30 g of Astragalus membranaceus (Fisch) Bge. Var. monghlicus (Bge) Hsiao, 10 g of Sophora flavescens Alt., 15 g of Ranunculus ternatus Thunb., 15 g of Salvia miltiorrhiza Bge., 30 g of Sedum sarmentosum Bunge and 30 g of Pueraria lobata (Willd.) Ohwi. The medicine materials were decocted with water for 2 times, and the volume of the water added for each time was 8 times relative to the weight of the medicine materials (V/W, L/kg), and decocted for 1.5 h. The decoction liquors were combined and filtrated, the filtrate was concentrated to give a dense extract which was further added with suitable amounts of microcrystalline cellulose, croscarmellose sodium and magnesium stearate and mixed uniformly. 5% PVPK30 solution was added with stirring to produce a soft material which was screened through a 16-mesh sieve for granulation, then tableted and coated to produce the tablets.

### Example 13: Preparation of Tablets

The medicine materials were weighed according to the following weights: 30 g of Morinda officinalis How, 15 g of Ganoderma lucidum (Leyss. Ex Fr.) Karst., 30 g of Astragalus membranaceus (Fisch) Bge. Var. monghlicus (Bge) Hsiao, 10 g of Sophora flavescens Alt., 15 g of Ranunculus ternatus Thunb., 15 g of Salvia miltiorrhiza Bge., 30 g of Sedum sarmentosum Bunge, 30 g of Pueraria lobata (Willd.) Ohwi, 30 g of Hovenia acerba Lindl., and 15 g of Flower of Pueraria lobata (Willd.) Ohwi. The medicine materials were decocted with water for 2 times, and the volume of the water added for each time was 8 times relative to the weight of the medicine materials (V/W, L/kg), and decocted for 1.5 h. The decoction liquors were combined and filtrated, the filtrate was concentrated to give a dense extract which was further added with suitable amounts of microcrystalline cellulose, croscarmellose sodium and magnesium stearate and mixed uniformly. 5% PVPK30 solution was added with stirring to produce a soft material which was screened through a 16-mesh sieve for granulation, then tableted and coated to produce the tablets.

### Example 14: Preparation of Tablets

The medicine materials were weighed according to the following weights: 30 g of Morinda officinalis How, 15 g of Ganoderma lucidum (Leyss. Ex Fr.) Karst., 30 g of Astragalus membranaceus (Fisch) Bge. Var. monghlicus (Bge) Hsiao, 10 g of Sophora flavescens Alt., 15 g of Ranunculus ternatus Thunb., 15 g of Salvia miltiorrhiza Bge., 15 g of Lycopus lucidus Turcz. var. hirtus Regel, 15 g of Curcuma wenyujin Y. H. Chen et C. Ling, 15 g of Stir-baked Trionyx sinensis Wiegmann, and 15 g of Dendrobium nobile Lindl. The medicine materials were decocted with water for 2 times, and the volume of the water added for each time was 8 times relative to the weight of the medicine materials (V/W, L/kg), and decocted for 1.5 h. The decoction liquors were combined and filtrated, the filtrate was concentrated to give a dense extract which was further added with suitable amounts of microcrystalline cellulose, croscarmellose sodium and magnesium stearate and mixed uniformly. 5% PVPK30 solution was added with stirring to produce a soft material which was screened through a 16-mesh sieve for granulation, then tableted and coated to produce the tablets.

### Example 15: Preparation of Tablets

The medicine materials were weighed according to the following weights: 30 g of Morinda officinalis How, 15 g of Ganoderma lucidum (Leyss. Ex Fr.) Karst., 30 g of Astragalus membranaceus (Fisch) Bge. Var. monghlicus (Bge) Hsiao, 10 g of Sophora flavescens Alt., 15 g of Ranunculus ternatus Thunb., 15 g of Salvia miltiorrhiza Bge., 6 g of Cremastra appendiculata Makino, 15 g of Abrus cantoniensis Hance, 6 g of Curcuma phaeocaulis Val., 15 g of Atractylodes macrocephala Koidz., 15 g of Scutellaria barbata D. Don. The medicine materials were decocted with water for 2 times, and the volume of the water added for each time was 8 times relative to the weight of the medicine materials (V/W, L/kg), and decocted for 1.5 h. The decoction liquors were combined and filtrated, the filtrate was concentrated to give a dense extract which was further added with suitable amounts of microcrystalline cellulose, croscarmellose sodium and magnesium stearate and mixed uniformly. 5% PVPK30 solution was added with stirring to produce a soft material which was screened through a 16-mesh sieve for granulation, then tableted and coated to produce the tablets.

### Example 16: Preparation of Capsules

The medicine materials were weighed according to the following weights: 30 g of Morinda officinalis How, 15 g of Ganoderma lucidum (Leyss. Ex Fr.) Karst., 30 g of Astragalus membranaceus (Fisch) Bge. Var. monghlicus (Bge) Hsiao, 15 g of Sophora flavescens Alt., 15 g of Ranunculus ternatus Thunb., 15 g of Salvia miltiorrhiza Bge., 15 g of Epimedium brevicornu Maxim., 10 g of Rehmannia glutinosa Libosch., 15 g of Atractylodes macrocephala Koidz. and 10 g of Citrus reticulata Blanco. The medicine materials were decocted with water for 2 times, and the volume of the water added for each time was 10 times relative to the weight of the medicine materials (V/W, L/kg), and decocted for 2 h. The decoction liquors were combined and filtrated, the filtrate was concentrated to give a dense extract which was subjected to low temperature drying, then added with suitable amounts of adjuvants and mixed uniformly, dried, and loaded into capsules to obtain the capsules.

### Example 17: Preparation of Capsules

The medicine materials were weighed according to the following weights: 30 g of Morinda officinalis How, 15 g of Ganoderma lucidum (Leyss. Ex Fr.) Karst., 30 g of Astragalus membranaceus (Fisch) Bge. Var. monghlicus (Bge) Hsiao, 10 g of Sophora flavescens Alt., 15 g of Ranunculus ternatus Thunb., 15 g of Salvia miltiorrhiza Bge., 30 g of Sedum sarmentosum Bunge and 30 g of Pueraria lobata (Willd.) Ohwi. The medicine materials were decocted with water for 2 times, and the volume of the water added for each time was 10 times relative to the weight of the medicine materials (V/W, L/kg), and decocted for 2 h. The decoction liquors were combined and filtrated, the filtrate was concentrated to give a dense extract which was subjected to low temperature drying, then added with suitable amounts of adjuvants and mixed uniformly, dried, and loaded into capsules to obtain the capsules.

### Example 18: Preparation of Capsules

The medicine materials were weighed according to the following weights: 30 g of Morinda officinalis How, 15 g of Ganoderma lucidum (Leyss. Ex Fr.) Karst., 30 g of Astragalus membranaceus (Fisch) Bge. Var. monghlicus (Bge) Hsiao, 10 g of Sophora flavescens Alt., 15 g of Ranunculus ternatus Thunb., 15 g of Salvia miltiorrhiza Bge., 30 g of Sedum sarmentosum Bunge, 30 g of Pueraria lobata (Willd.) Ohwi, 30 g of Hovenia acerba Lindl., and 15 g of Flower of Pueraria lobata (Willd.) Ohwi. The medicine materials were decocted with water for 2 times, and the volume of the water added for each time was 10 times relative to the weight of the medicine materials (V/W, L/kg), and decocted for 2 h. The decoction liquors were combined and filtrated, the filtrate was concentrated to give a dense extract which was subjected to low temperature drying, then added with suitable amounts of adjuvants and mixed uniformly, dried, and loaded into capsules to obtain the capsules.

### Example 19: Preparation of Capsules

The medicine materials were weighed according to the following weights: 30 g of Morinda officinalis How, 15 g of Ganoderma lucidum (Leyss. Ex Fr.) Karst., 30 g of Astragalus membranaceus (Fisch) Bge. Var. monghlicus (Bge) Hsiao, 10 g of Sophora flavescens Alt., 15 g of Ranunculus ternatus Thunb., 15 g of Salvia miltiorrhiza Bge., 15 g of Lycopus lucidus Turcz. var. hirtus Regel, 15 g of Curcuma wenyujin Y. H. Chen et C. Ling, 15 g of Stir-baked Trionyx sinensis Wiegmann, and 15 g of Dendrobium nobile Lindl. The medicine materials were decocted with water for 2 times, and the volume of the water added for each time was 10 times relative to the weight of the medicine materials (V/W, L/kg), and decocted for 2 h. The decoction liquors were combined and filtrated, the filtrate was concentrated to give a dense extract which was subjected to low temperature drying, then added with suitable amounts of adjuvants and mixed uniformly, dried, and loaded into capsules to obtain the capsules.

### Example 20: Preparation of Capsules

The medicine materials were weighed according to the following weights: 30 g of Morinda officinalis How, 15 g of Ganoderma lucidum (Leyss. Ex Fr.) Karst., 30 g of Astragalus membranaceus (Fisch) Bge. Var. monghlicus (Bge) Hsiao, 10 g of Sophora flavescens Alt., 15 g of Ranunculus ternatus Thunb., 15 g of Salvia miltiorrhiza Bge., 6 g of Cremastra appendiculata Makino, 15 g of Abrus cantoniensis Hance, 6 g of Curcuma phaeocaulis Val., 15 g of Atractylodes macrocephala Koidz., 15 g of Scutellaria barbata D. Don. The medicine materials were decocted with water for 2 times, and the volume of the water added for each time was 10 times relative to the weight of the medicine materials (V/W, L/kg), and decocted for 2 h. The decoction liquors were combined and filtrated, the filtrate was concentrated to give a dense extract which was subjected to low temperature drying, then added with suitable amounts of adjuvants and mixed uniformly, dried, and loaded into capsules to obtain the capsules.

### Example 21: Preparation of the Compound Dry Extract

The medicine materials were weighed according to the following weights: 30 g of Morinda officinalis How, 15 g of Ganoderma lucidum (Leyss. Ex Fr.) Karst., 30 g of Astragalus membranaceus (Fisch) Bge. Var. monghlicus (Bge) Hsiao, 15 g of Sophora flavescens Alt., 15 g of Ranunculus ternatus Thunb., 15 g of Salvia miltiorrhiza Bge., 15 g of Epimedium brevicornu Maxim., 10 g of Rehmannia glutinosa Libosch., 15 g of Atractylodes macrocephala Koidz. and 10 g of Citrus reticulata Blanco. The medicine materials were mixed and comminuted, then screened through a 20-mesh sieve, followed by decoction with water for 2 times, and the volume of the water added for each time was 15 times relative to the weight of the medicine materials (V/W, L/kg). The decoction liquor was filtrated, the filtrate was centrifuged and the supernatant was concentrated to provide a clear extract. The clear extract was subjected to freeze drying or spray drying to obtain the compound dry extract of water decoction liquors.

### Example 22: Preparation of the Compound Dry Extract

The medicine materials were weighed according to the following weights: 30 g of Morinda officinalis How, 15 g of Ganoderma lucidum (Leyss. Ex Fr.) Karst., 30 g of Astragalus membranaceus (Fisch) Bge. Var. monghlicus (Bge) Hsiao, 10 g of Sophora flavescens Alt., 15 g of Ranunculus ternatus Thunb., 15 g of Salvia miltiorrhiza Bge., 30 g of Sedum sarmentosum Bunge and 30 g of Pueraria lobata (Willd.) Ohwi. The medicine materials were mixed and comminuted, then screened through a 20-mesh sieve, followed by decoction with water for 2 times, and the volume of the water added for each time was 15 times relative to the weight of the medicine materials (V/W, L/kg). The decoction liquor was filtrated, the filtrate was centrifuged and the supernatant was concentrated to provide a clear extract. The clear extract was subjected to freeze drying to obtain the compound dry extract of water decoction liquors.

### Example 23: Preparation of the Compound Dry Extract

The medicine materials were weighed according to the following weights: 30 g of Morinda officinalis How, 15 g of Ganoderma lucidum (Leyss. Ex Fr.) Karst., 30 g of Astragalus membranaceus (Fisch) Bge. Var. monghlicus (Bge) Hsiao, 10 g of Sophora flavescens Alt., 15 g of Ranunculus ternatus Thunb., 15 g of Salvia miltiorrhiza Bge., 30 g of Sedum sarmentosum Bunge, 30 g of Pueraria lobata (Willd.) Ohwi, 30 g of Hovenia acerba Lindl., and 15 g of Flower of Pueraria lobata (Willd.) Ohwi. The medicine materials were mixed and comminuted, then screened through a 20-mesh sieve, followed by decoction with water for 2 times, and the volume of the water added for each time was 15 times relative to the weight of the medicine materials (V/W, L/kg). The decoction liquor was filtrated, the filtrate was centrifuged (3000 r/min) and the supernatant was concentrated to provide a clear extract. The clear extract was subjected to freeze drying to obtain the compound dry extract of water decoction liquors.

### Example 24: Preparation of the Compound Dry Extract

The medicine materials were weighed according to the following weights: 30 g of Morinda officinalis How, 15 g of Ganoderma lucidum (Leyss. Ex Fr.) Karst., 30 g of Astragalus membranaceus (Fisch) Bge. Var. monghlicus (Bge) Hsiao, 10 g of Sophora flavescens Alt., 15 g of Ranunculus ternatus Thunb., 15 g of Salvia miltiorrhiza Bge., 15 g of Epimedium brevicornu Maxim., 10 g of Rehmannia glutinosa Libosch., 30 g of Atractylodes macrocephala Koidz. and 9 g of Citrus reticulata Blanco. The medicine materials were mixed and comminuted, then screened through a 20-mesh sieve, followed by decoction with water for 2 times, and the volume of the water added for each time was 15 times relative to the weight of the medicine materials (V/W, L/kg). The decoction liquor was filtrated, the filtrate was centrifuged (3000 r/min) and the supernatant was concentrated to provide a clear extract. The clear extract was subjected to freeze drying to obtain the compound dry extract of water decoction liquors.

### Example 25: Preparation of the Compound Dry Extract

The medicine materials were weighed according to the following weights: 30 g of Morinda officinalis How, 15 g of Ganoderma lucidum (Leyss. Ex Fr.) Karst., 30 g of Astragalus membranaceus (Fisch) Bge. Var. monghlicus (Bge) Hsiao, 10 g of Sophora flavescens Alt., 15 g of Ranunculus ternatus Thunb., 15 g of Salvia miltiorrhiza Bge., 6 g of Cremastra appendiculata Makino, 15 g of Abrus cantoniensis Hance, 6 g of Curcuma phaeocaulis Val., 15 g of Atractylodes macrocephala Koidz., 15 g of Scutellaria barbata D. Don. The medicine materials were mixed and comminuted, then screened through a 20-mesh sieve, followed by decoction with water for 2 times, and the volume of the water added for each time was 15 times relative to the weight of the medicine materials (V/W, L/kg). The decoction liquor was filtrated, the filtrate was centrifuged (3000 r/min) and the supernatant was concentrated to provide a clear extract. The clear extract was subjected to freeze drying to obtain the compound dry extract of water decoction liquors.

### Example 26: Preparation of the Compound Dry Extract of the Alcohol Precipitation Supernatant and Preparation of the Compound Polysaccharide

The medicine materials were weighed according to the following weights: 30 g of Morinda officinalis How, 15 g of Ganoderma lucidum (Leyss. Ex Fr.) Karst., 30 g of Astragalus membranaceus (Fisch) Bge. Var. monghlicus (Bge) Hsiao, 15 g of Sophora flavescens Alt., 15 g of Ranunculus ternatus Thunb., 15 g of Salvia miltiorrhiza Bge., 15 g of Epimedium brevicornu Maxim., 10 g of Rehmannia glutinosa Libosch., 15 g of Atractylodes macrocephala Koidz. and 10 g of Citrus reticulata Blanco. The medicine materials were comminuted and screened through a 20-mesh sieve, followed by decoction with water for 2 times, and the volume of the water added for each time was 15 times relative to the weight of the medicine materials (V/W, L/kg). The decoction liquor was filtrated, the filtrate was centrifuged and the supernatant was concentrated. The obtained concentrated liquor was added with ethanol, volume of which was 3 times (V/V) relative to the volume of the obtained concentrated liquor and allowed to stand still for precipitation for 72 hours, then centrifuged. The alcohol precipitation supernatant was concentrated under reduced pressure and dried in vacuum to produce the compound dry extract of alcohol precipitation supernatant. The precipitation fraction was dissolved by adding water for many times, and the obtained solution was charged into a dialysis bag to conduct water dialysis for 48 hours (to intercept fraction with molecular weight >1000 Da). The solution in the bag was concentrated and freeze dried to produce the compound polysaccharide.

### Example 27: Preparation of the Compound Dry Extract of the Alcohol Precipitation Supernatant and Preparation of the Compound Polysaccharide

The medicine materials were weighed according to the following weights: 30 g of Morinda officinalis How, 15 g of Ganoderma lucidum (Leyss. Ex Fr.) Karst., 30 g of Astragalus membranaceus (Fisch) Bge. Var. monghlicus (Bge) Hsiao, 10 g of Sophora flavescens Alt., 15 g of Ranunculus ternatus Thunb., 15 g of Salvia miltiorrhiza Bge., 30 g of Sedum sarmentosum Bunge, and 30 g of Pueraria lobata (Willd.) Ohwi. The medicine materials were comminuted and screened through a 20-mesh sieve, followed by decoction with water for 2 times, and the volume of the water added for each time was 15 times relative to the weight of the medicine materials (V/W, L/kg). The decoction liquor was filtrated, the filtrate was centrifuged and the supernatant was concentrated. The obtained concentrated liquor was added with ethanol, volume of which was 3 times (V/V) relative to the volume of the obtained concentrated liquor, and allowed to stand still for precipitation for 72 hours, then centrifuged. The alcohol precipitation supernatant was concentrated under reduced pressure and dried in vacuum to produce the compound dry extract of alcohol precipitation supernatant. The precipitation fraction was dissolved by adding water for many times, and the obtained solution was charged into a dialysis bag to conduct water dialysis for 48 hours (to intercept fraction with molecular weight >1000 Da). The solution in the bag was concentrated and freeze dried to produce the compound polysaccharide.

### Example 28: Preparation of the Compound Dry Extract of the Alcohol Precipitation Supernatant and Preparation of the Compound Polysaccharide

The medicine materials were weighed according to the following weights: 30 g of Morinda officinalis How, 15 g of Ganoderma lucidum (Leyss. Ex Fr.) Karst., 30 g of Astragalus membranaceus (Fisch) Bge. Var. monghlicus (Bge) Hsiao, 10 g of Sophora flavescens Alt., 15 g of Ranunculus ternatus Thunb., 15 g of Salvia miltiorrhiza Bge., 30 g of Sedum sarmentosum Bunge, 30 g of Pueraria lobata (Willd.) Ohwi, 30 g of Hovenia acerba Lindl., and 15 g of Flower of Pueraria lobata (Willd.) Ohwi. The medicine materials were comminuted and screened through a 20-mesh sieve, followed by decoction with water for 2 times, and the volume of the water added for each time was 15 times relative to the weight of the medicine materials (V/W, L/kg). The decoction liquor was filtrated, the filtrate was centrifuged (3000 r/min) and the supernatant was concentrated. The obtained concentrated liquor was added with ethanol, volume of which was 3 times (V/V) relative to the volume of the obtained concentrated liquor, and allowed to stand still for precipitation for 72 hours, then centrifuged. The alcohol precipitation supernatant was concentrated under reduced pressure and dried in vacuum to produce the compound dry extract of alcohol precipitation supernatant. The precipitation fraction was dissolved by adding water for many times, and the obtained solution was charged into a dialysis bag to conduct water dialysis for 48 hours (to intercept fraction with molecular weight >1000 Da). The solution in the bag was concentrated and freeze dried to produce the compound polysaccharide.

### Example 29: Preparation of the Compound Dry Extract of the Alcohol Precipitation Supernatant and Preparation of the Compound Polysaccharide

The medicine materials were weighed according to the following weights: 30 g of Morinda officinalis How, 15 g of Ganoderma lucidum (Leyss. Ex Fr.) Karst., 30 g of Astragalus membranaceus (Fisch) Bge. Var. monghlicus (Bge) Hsiao, 10 g of Sophora flavescens Alt., 15 g of Ranunculus ternatus Thunb., 15 g of Salvia miltiorrhiza Bge., 15 g of Lycopus lucidus Turcz. var. hirtus Regel, 15 g of Curcuma wenyujin Y. H. Chen et C. Ling, 15 g of Stir-baked Trionyx sinensis Wiegmann, and 15 g of Dendrobium nobile Lindl. The medicine materials were comminuted and screened through a 20-mesh sieve, followed by decoction with water for 2 times, and the volume of the water added for each time was 15 times relative to the weight of the medicine materials (V/W, L/kg). The decoction liquor was filtrated, the filtrate was centrifuged (3000 r/min) and the supernatant was concentrated. The obtained concentrated liquor was added with ethanol, volume of which was 3 times (V/V) relative to the volume of the obtained concentrated liquor, and allowed to stand still for precipitation for 72 hours, then centrifuged. The alcohol precipitation supernatant was concentrated under reduced pressure and dried in vacuum to produce the compound dry extract of alcohol precipitation supernatant. The precipitation fraction was dissolved by adding water for many times, and the obtained solution was charged into a dialysis bag to conduct water dialysis for 48 hours (to intercept fraction with molecular weight >1000 Da). The solution in the bag was concentrated and freeze dried to produce the compound polysaccharide.

### Example 30: Preparation of the Compound Dry Extract of the Alcohol Precipitation Supernatant and Preparation of the Compound Polysaccharide

The medicine materials were weighed according to the following weights: 30 g of Morinda officinalis How, 15 g of Ganoderma lucidum (Leyss. Ex Fr.) Karst., 30 g of Astragalus membranaceus (Fisch) Bge. Var. monghlicus (Bge) Hsiao, 10 g of Sophora flavescens Alt., 15 g of Ranunculus ternatus Thunb., 15 g of Salvia miltiorrhiza Bge., 6 g of Cremastra appendiculata Makino, 15 g of Abrus cantoniensis Hance, 6 g of Curcuma phaeocaulis Val., 15 g of Atractylodes macrocephala Koidz., and 15 g of Scutellaria barbata D. Don. The medicine materials were comminuted and screened through a 20-mesh sieve, followed by decoction with water for 2 times, and the volume of the water added for each time was 15 times relative to the weight of the medicine materials (V/W, L/kg). The decoction liquor was filtrated, the filtrate was centrifuged (3000 r/min) and the supernatant was concentrated. The obtained concentrated liquor was added with ethanol, volume of which was 3 times (V/V) relative to the volume of the obtained concentrated liquor and allowed to stand still for precipitation for 72 hours, then centrifuged. The alcohol precipitation supernatant was concentrated under reduced pressure and dried in vacuum to produce the compound dry extract of alcohol precipitation supernatant. The precipitation fraction was dissolved by adding water for many times, and the obtained solution was charged into a dialysis bag to conduct water dialysis for 48 hours (to intercept fraction with molecular weight >1000 Da). The solution in the bag was concentrated and freeze dried to produce the compound polysaccharide.

### Example 31: Evaluations of Curative Effects on Patients with Hepatitis B

1. Test Sample: The Granules Prepared According to the Method of Example 6

2. Evaluation Method

The cases were patients with HBeAg negative chronic hepatitis B, from hepatology clinic of Shuguang Hospital affiliated to Shanghai University of Traditional Chinese Medicine in the period from December, 2012 to March, 2016 and in a total number of 136, with 89 males and 47 females, in ages of 18-64 years, averaging 43.98 years. All the cases met the diagnostic standards of chronic hepatitis B in 2010, and having HBV-DNA ≥10⁴ and HBeAg negativity. According to traditional Chinese medical symptom differentiations, the cases had symptoms of liver and kidney deficiency and damp heat, and as for the corresponding standards, please see Guidelines for Clinical Researches on New Traditional Chinese Medicines. The cases were randomly divided into the treatment group and control group by applying randomized controlled trial method in a ratio of 1:1, wherein the treatment group included 68 cases, the control group included 68 cases, and there were no difference of the two groups in terms of age-sex pattern and disease course.

Dosage regimen: both the treatment group and the control group were subjected to antiviral treatment and traditional Chinese medicine treatment simultaneously, with a treatment course of 30 months.

For the antiviral treatment: both the treatment group and the control group were administered with Entecavir dispersible tablets (0.5 mg/tablet, 1 tablet/time, 1 time/day, empty stomach, oral administration) to conduct antiviral treatment; for the traditional Chinese medicine treatment: patients in the treatment group were administered with the test sample (in a dosage calculated based on the clear extract prepared in the example, 60 g clear extract/day, and administrated twice with lukewarm water, once in the morning and once in the evening); the patients in the control group were administered with placebos (granules, a drug-free simulated formulation) in the same manner as the treatment group.

3. Results

A total of 136 patients were enrolled in this study, with 68 patients in the treatment group and 68 patients in the control group, and for all, 30-month follow-up visit was finished. 79 patients underwent liver biopsy before treatment, and 43 patients underwent liver biopsy after treatment, wherein 40 patients underwent the two times liver biopsies before and after the treatment. At the time point of 24 weeks, 7 patients in the control group were out of touch, and 1 patient in the treatment group was out of touch. 120 patients completed a 96-week follow-up visit, wherein 60 patients were in the treatment group, and 60 patients were in the control group. For those completing the 30-month follow-up visit, 59 patients were in the control group, and 56 patients were in the treatment group.

After 30-month treatment, the patients were bled to determine serum ALT and AST values to calculate the recurrence rate. The ALT recurrence rate of the control group was 93.2%, and the recurrence rate of the treatment group was 98.2%. The HBsAg of the treatment group was averagely reduced by 0.187 log IU/ml, and the HBsAg of the control group was averagely reduced by 0.028 log IU/ml, and the reduction extent of the treatment group was higher than that of the control group.

According to the symptom data recorded by the CRF chart, the first 14 symptoms were analyzed, wherein the symptoms with the highest incidence rate, in order, were lassitude, asthenopia, soreness and weakness of waist and knees, impatience, dry eyes, hypochondriac pain, insomnia and dreaminess, bitter taste, tinnitus, dizziness, dim complexion, sighing, yellow skill and eyes, and dysphoria in chestpalms-soles. The lassitude had the highest incidence rate, accounting for 46%; the incidence rate of the asthenopia took the second place, accounting for 42%; the incidence rate of the soreness and weakness of waist and knees was 40%; the incidence rate of the impatience and dysphoria was 38%; the incidence rate of the dry eyes symptom was 37%; the incidence rate of the hypochondriac pain was 36%; the incidence rates of insomnia, bitter taste, tinnitus, dizziness, dim complexion, sighing, yellow skin and eyes, and dysphoria in chestpalms-soles were respectively 34%, 20%, 19%, 16%, 15%, 7%, 5% and 3%. After treatment, the improvement of symptoms including asthenopia, insomnia and dreaminess, and lassitude of the patients in the treatment group was significantly better than those in the control group, the difference was statistically significant (P<0.05).

### Example 32: Evaluation of Curative Effect on Patients with Nonalcoholic Steatohepatitis

1. Test Sample: The Granules Prepared According to the Method in Example 7

2. Method

A randomized controlled trial method was applied. A total of 88 patients with non-alcoholic steatohepatitis were enrolled and randomly divided into the treatment group (44 patients) and control group (44 patients). The two groups were both subjected to diet and exercise interventions, on the basis of that, the treatment group was administrated with the test sample (in a dosage calculated based on the clear extract prepared in the example, 60 g clear extract/day, and administrated twice), and the control group was administrated with placebos (a drug-free simulated formulation), each with a treatment course of 12 weeks. Changes in traditional Chinese medical symptom scores, liver functions, blood fat and imaging indexes (liver/spleen CT values) of the patients before and after treatment were observed.

3. Results

(1) After treatment, the patients in the treatment group were bled to measure serum ALT, AST, GGT, TG and total cholesterol levels by using biochemical analyzer. The results showed that all indexes of the treatment group were remarkably reduced compared to those before treatment (P<0.05 or P<0.01), and the differences between the treatment group and the control group after the treatment were statistically significant (P<0.01). Specific results were shown in Table 1.

TABLE 1
Changes in liver functions and blood fat levels of patients in the two groups before and after treatment (x ± s)

| Group | | ALT (U/L) | AST (U/L) | GGT (U/L) | TCh (mmol/L) | TG (mmol/L) |
|---|---|---|---|---|---|---|
| Treatment | Before | 82.2 ± 24.8 | 50.0 ± 18.9 | 60.4 ± 36.0 | 5.46 ± 1.13 | 2.21 ± 1.28 |
| group | treatment | | | | | |
| | After | 46.1 ± 22.7^{#}* | 30.5 ± 8.3^{#}* | 42.6 ± 22.0^{#}* | 5.14 ± 1.11^{##}* | 2.47 ± 2.25 |
| | treatment | | | | | |
| Control | Before | 76.2 ± 25.2 | 43.9 ± 20.8 | 47.2 ± 33.7 | 5.24 ± 0.83 | 2.19 ± 1.06 |
| group | treatment | | | | | |
| | After | 62.7 ± 35.1 | 41.0 ± 19.1 | 48.2 ± 23.8 | 5.22 ± 0.93 | 2.37 ± 1.34 |
| | treatment | | | | | |

Note:
compared with the same group before treatment,
^{#}P < 0.05,
^{#}P < 0.01;
compared with the control group after treatment,
*P < 0.05.

(2) After treatment, the liver/spleen CT values of the patients in the treatment group were obviously increased, the grade was obviously improved (P<0.01), the differences between the two groups after treatment were statistically significant, and the curative effect of the treatment group was better than that of the control group (P<0.01). Specific data were shown in Table 2.

TABLE 2
Changes in liver/spleen CT grades of patients
in the two groups before and after treatment

| | Group | | | | | | | | |
|---|---|---|---|---|---|---|---|---|---|
| | Before treatment | | | | After treatment | | | | |
| Grade | 0 | Mild | Moderate | Severe | 0 | Mild | Moderate | Severe | P value |
| Treatment group | 0 | 23 | 11 | 10 | 16 | 21 | 6 | 1 | 0.001 |
| (person number) | | | | | | | | | |
| Control group | 0 | 24 | 13 | 9 | 1 | 12 | 12 | 9 | 0.291 |
| (person number) | | | | | | | | | |

### Example 33: Evaluation of Curative Effect on Patients with Alcoholic Steatohepatitis

1. Test Sample: The Granules Prepared According to the Method in Example 8

2. Method

A randomized controlled trial method was applied. A total of 46 alcoholic steatohepatitis patients were enrolled and were randomly divided into treatment group (23 patients) and control group (23 patients). The two groups were both subjected to diet and exercise interventions, on the basis of that, the treatment group was administrated with the test sample (in a dosage calculated based on the clear extract prepared in the example, 60 g clear extract/day, and administrated twice), and the control group was administrated with placebos (a drug-free simulated formulation), each with a treatment course of 12 weeks. Changes in traditional Chinese medical symptom scores, liver functions, blood fat and imaging indexes (liver/spleen CT values) of the patients before and after treatment were observed.

3. Results

After treatment, the serum ALT, AST, GGT, and total cholesterol levels of the patients in the treatment group were remarkably decreased (P<0.05 or P<0.01), and the differences between the two groups after treatment were statistically significant (P<0.01). Acratia and Anorexia in traditional Chinese Medical symptoms of the treatment group were improved significantly.

### Example 34: Evaluation of Curative Effect on Patients with Hepatic Fibrosis

1. Test Sample: The Granules Prepared According to the Method in Example 9

2. Method

A Randomized grouping, double-blind, placebo-control, and multicenter clinical trial method was applied. A total of 400 CHB patients (95 patients underwent liver biopsy before and after treatment) were enrolled and randomly divided into treatment group and control group. The treatment group was administrated with Entecavir in combination with the test sample (in a dosage calculated based on the clear extract prepared in the example, 60 g clear extract/day, and administrated twice), and the control group was administrated with Entecavir in combination with placebo (a drug-free simulated formulation), each with a treatment course of 12 months. Changes in HBeAg levels and liver pathological inflammation and fibrosis of the patients in the two groups were observed.

3. Results

(1) After treatment, the HBeAg levels of the patients in the two groups were remarkably decreased as compared to those before treatment (P<0.001), and the HBeAg level of the treatment group was lower than that of the control group (P<0.05), and the reduction extent of the HBeAg level of the treatment group was significantly superior to that of the control group (P<0.01).

(2) After treatment, the proportion of the patients with a liver inflammation grade of ≥G2 in the treatment group was reduced from 100% to 66.67%, and the proportion of the patients with a fibrosis staging grade ≥S3 was reduced from 27.45% to 15.69%. Moreover, the improvement in the fibrosis stage was significantly superior to that of the control group administrated with Entecavir alone (P<0.05).

### Example 35: Evaluation of Curative Effect on Patients with Compensated Cirrhosis

1. Test Sample: The Granules Prepared According to the Method in Example 9

2. Method

A prospective cohort study was conducted. A total of 126 patients with compensated hepatitis B cirrhosis were enrolled, wherein the group of western medicine+the test sample was administrated with the test sample (in a dosage calculated based on the clear extract prepared in the example, 60 g clear extract/day, and administrated twice) in combination with Entecavir (0.5 mg/day), the test sample group was administrated with the test sample alone (in a dosage calculated based on the clear extract prepared in the example, 60 g clear extract/day, and administrated twice), and the control group was administered with Compound Biejia Ruangan Tablets, each with a treatment course of 12 months. Traditional Chinese medicine symptom scores, Child-Pugh liver function grades (CTP scores), chronic liver disease specific qualifying (CLDQ) scale scores, liver functions, and pathological changes through liver biopsy before and after treatment were observed and compared.

3. Results

(1) After treatment, CTP scores of patients with hepatitis B cirrhosis were remarkably decreased as compared to those before treatment, and the difference of the two groups before and after treatment were statistically significant (P<0.05), and the curative effect of the treatment group was superior to that of the control group (P<0.05) (see Table 3).

TABLE 3
Changes in Child-Pugh grades (CTP scores) of patients with compensated
hepatitis B cirrhosis before and after treatment

| | | Before | After |
|---|---|---|---|
| Group | | treatment | treatment |
| Western medicine + | Treatment group | 5.61 ± 0.65 | 5.03 ± 0.35^{#}* |
| test sample | Control group | 5.75 ± 0.67 | 5.13 ± 0.47^{#}* |
| Test sample | Treatment group | 5.63 ± 0.83 | 5.37 ± 0.51^{##}* |
| | Control group | 5.61 ± 0.58 | 5.50 ± 0.52^{#} |

Note:
compared with that before treatment, ^{#}P < 0.05, ^{##}P < 0.01;
compared with that in the control group, *P < 0.05

(2) After treatment, the CLDQ score of patients with compensated hepatitis B cirrhosis was obviously reduced as compared to that before treatment (P<0.01), and the difference of the two groups after antiviral treatment was statistically significant, and the curative effect was better than that of the control group (P<0.05), as shown in Table 4.

TABLE 4
Changes in CLDQ scores of patients with compensated
hepatitis B cirrhosis before and after treatment

| | | Before | After |
|---|---|---|---|
| Group | | treatment | treatment |
| Western medicine + | Treatment group | 56.8 ± 11.7 | 37.2 ± 3.6^{#} |
| test sample | Control group | 58.6 ± 12.0 | 45.9 ± 10.5^{#}* |
| Test sample | Treatment group | 59.7 ± 6.3 | 41.0 ± 5.5^{##}* |
| | Control group | 56.4 ± 6.4 | 5.50 ± 0.52^{#} |

Note:
compared with that before treatment, ^{##}P < 0.01;
compared with that in the control group, *P < 0.05

### Example 36: Evaluation of Curative Effect of the Compound Granules on Patients with Liver Cancer

1. Test Sample: The Granules Prepared According to the Method in Example 10

2. Method

The research started from September 2014 to March 2017, according to inclusion criteria, a total of 210 patients with advanced primary hepatocarcinoma after surgery from Shuguang Hospital affiliated to Shanghai University of Traditional Chinese Medicine, Changhai Hospital of Shanghai, and Zhongshan Hosptical affiliated to Fudan University were enrolled, wherein 18 patients were out of touch and 12 patients were rejected during research, and 180 patients were observed finally.

According to Diagnosis, management, and treatment of hepatocellular carcinoma (V2011) issued by Ministry of Health of the People's Republic of China, the patients with advanced primary hepatocarcinoma (patients that were in Stage B and Stable C according to BCLC staging) after surgery (operation, intervention or radiofrequency ablation) signed Informed Consent Form, and were divided into control group and treatment group. The control group was administrated with Entecavir (0.5 mg/day), and the treatment group was administrated with the test sample (in a dosage calculated based on the clear extract prepared in the example, 60 g clear extract/day, and administrated twice) in combination with Entecavir (0.5 mg/day). The patients in the two groups were observed in a period of 6 months.

3. Results

By analyzing and comparing the accumulative survival rates of the patients in the two groups, the accumulative survival rate of the treatment group is 88.9%, while that of the control group is 76.7%, the survival rate of the two groups is significantly different from each other (P<0.05). During the treatment and the follow-up observation, at 3 months of treatment, 3 deaths occurred in each of the treatment and control group; at 6 months of treatment, 5 deaths occurred in the treatment group and 9 deaths occurred in the control group; at 3 months of follow-up visit, 10 deaths occurred in the treatment group and 17 deaths occurred in the control group; at 6 months of follow-up visit, 10 deaths occurred in the treatment group, and 21 deaths occurred in the control group; the fatality rate of the control group was significantly higher than that of the treatment group (P<0.05).

At 3 months of treatment, 8 patients of liver cancer recurred in the treatment group and 13 patients in the control group; at 6 months of treatment, 30 patients recurred in the treatment group and 44 patients in the control group; there is a statistical differences (P<0.05) in the recurrence rate between the two groups; at 3 months of follow-up visit, 32 patients recurred in the treatment group and 48 patients in the control group; there is a statistical differences (P<0.05) in the recurrence rate of liver cancer between the two groups.

In terms of improving main traditional Chinese medicine symptoms in patients with primary hepatocarcinoma after surgery, 42 cases among 61 patients with tastelessness in the treatment group were improved, while in the control group, 25 cases among 58 patients with tastelessness were improved. There was significant difference between the two groups (P<0.05). 53 cases among 68 patients with stomach distension in the treatment group were improved, while in the control group, 35 cases among 62 patients were improved. There was significant difference between the two groups (P<0.05). 20 cases among 38 patients with nausea and vomiting in the treatment group were improved, while in the control group, 15 cases among 51 patients were improved. There was significant difference between the two groups (P<0.05). 62 cases among 73 patients with fatigue and acratia in the treatment group were improved, while in the control group, 45 cases among 69 patients were improved. There was significant difference between the two groups (P<0.05). In therapeutic effects on traditional Chinese medicine symptoms, after treatment, in the treatment group, there were 2 patients with curative effect, 53 valid cases and 30 invalid cases; in the control group, there was 0 patient with curative effect, 16 valid cases and 65 invalid cases. There was significant difference in the therapeutic effects on traditional Chinese medicine symptoms between the two groups (P<0.01).

### Example 37: Evaluation on the Activity Against Liver Injury

1. Test Sample: The Compound Dry Extract Prepared According to Example 21

2. Method

40 female Balb/C mice (purchased from Beijing Charles River company) were randomly divided into four groups, 10 in each group: (1) normal control group; (2) ConA-induced liver injury model group; (3) compound dry extract group (2.0 g/kg); and (4) compound glycyrrhizin tablet (Beijing Kawin Technology Share-Holding Co., Ltd., 50 mg/kg) group. The mice in the model group were injected with ConA twice in a week into tail vein, in a dosage of 10 mg/kg for each time, and the injection were continued for 2 weeks. The compound dry extract group and the compound glycyrrhizin tablet group were respectively intragastrically administrated the ConA-induced liver injury model mice with the compound dry extract (2.0 g/kg) and the compound glycyrrhizic acid tablets (50 mg/kg) for once per day, and the administration was continued for 2 weeks. The normal control group was intragastrially administrated with physiological saline for once per day, and the administration was continued for 2 weeks. After the last ConA injection and drug administration, the mice were fasted overnight, then sacrificed. Spleens of the mice were aseptically taken out to prepare the splenocyte suspension, and effect of the compound dry extract on mouse splenocyte proliferation was determined; the livers, kidneys and thymus of the mice were taken out and weighed, and weight index (organ weight/weight of mouse body) was calculated. Pathological slices of liver and kidney were prepared to observe pathological injury of the liver and kidney.

Method of Experiment of Spleen Lymphocyte Proliferation

The spleen was taken out under aseptic condition to prepare splenocyte suspension, and the cell survival rate of >95% was determined by trypan blue staining method, and cell counting was performed to adjust cell density to 5×10⁶/mL. The counted splenocyte suspension was added into 96-well cell culture plate in 100 μL per well, and solvent control wells were only added with equivalent volume of RPMI1640 culture solution as background. Each well was further added with 100 μL of the test sample (with the final concentrations of 10, 50 and 250 μg/mL, respectively), ConA (with the final concentration of 2 μg/mL) and LPS (with the final concentration of 15 μg/mL), triplicate wells were set up for each group. The culture plate was placed in an incubator with 5% CO₂, and incubated at 37° C. for 72 h, then added with 20 μL of MTT 5 μg/mL, the incubation was continued for 4 h. After the supernatant was discarded, each well was added with 150 μL of DMSO, and enzyme-labelled meter was used to detect the OD value at A_{570 nm} to calculate the cell proliferation rate.

3. Results

(1) Compared to the control group, livers and kidneys of the mice in ConA-inducted liver injury model group enlarged, weight index (P<0.01) increased significantly, thymus atrophied, and weight index reduced. Administrating the compound dry extract could remarkably improve liver enlargement (P<0.05), and could also improve relieving kidney enlargement and thymus atrophy to some extent (see Table 5).

TABLE 5
Effect of the compound dry extract on organ weight of mice with liver injury

| | Dosage | Liver weight | Kidney weight | Thymus weight |
|---|---|---|---|---|
| Group | (g/kg) | index (10⁻¹) | index (10⁻¹) | index (10⁻¹) |
| Normal control | | 4.052 ± 0.239 | 1.197 ± 0.086 | 0.189 ± 0.067 |
| group | | | | |
| Model group | | 5.175 ± 0.373** | 1.403 ± 0.161** | 0.130 ± 0.070 |
| Compound dry | 2.0 | 4.564 ± 0.257^{#} | 1.300 ± 0.066 | 0.163 ± 0.031 |
| extract group | | | | |
| Compound | 0.05 | 4.730 ± 0.467 | 1.278 ± 0.183 | 0.130 ± 0.081 |
| glycyrrhizin | | | | |
| tablet group | | | | |

Note:
as compared to the normal group, *P < 0.05, **P < 0.01; as compared to the model group, ^{#}P < 0.05, n = 10.

(2) The compound dry extract could promote splenocyte proliferation (P<0.05), and it also had apparent synergistic effect on ConA- and LPS-stimulated T cell and B cell proliferations (P<0.001).

TABLE 6
Effect of the compound dry extract on splenocyte
proliferation of mice with liver injury

| | Dosage | OD_{570 nm} | | |
|---|---|---|---|---|
| Group | (g/kg) | RPMI1640 | ConA | LPS |
| Normal | — | 0.102 ± 0.007 | 0.182 ± 0.014 | 0.200 ± 0.011 |
| control group | | | | |
| ConA | 0.01 | | 0.348 ± 0.020*** | |
| LPS | | | | 0.503 ± 0.104*** |
| Compound | 2.0 | 0.245 ± 0.058** | 0.445 ± 0.018^{###} | 0.797 ± 0.049^{###} |
| dry extract | | | | |
| group | | | | |
| Compound | 0.05 | 0.209 ± 0.006* | 0.354 ± 0.012 | 0.497 ± 0.013 |
| glycyrrhizin | | | | |
| tablet group | | | | |

Note:
as compare to the normal group, *P < 0.05, **P < 0.01;
as compared to the model group, ^{#}P < 0.05, ^{##}P < 0.01, ^{###}P < 0.001

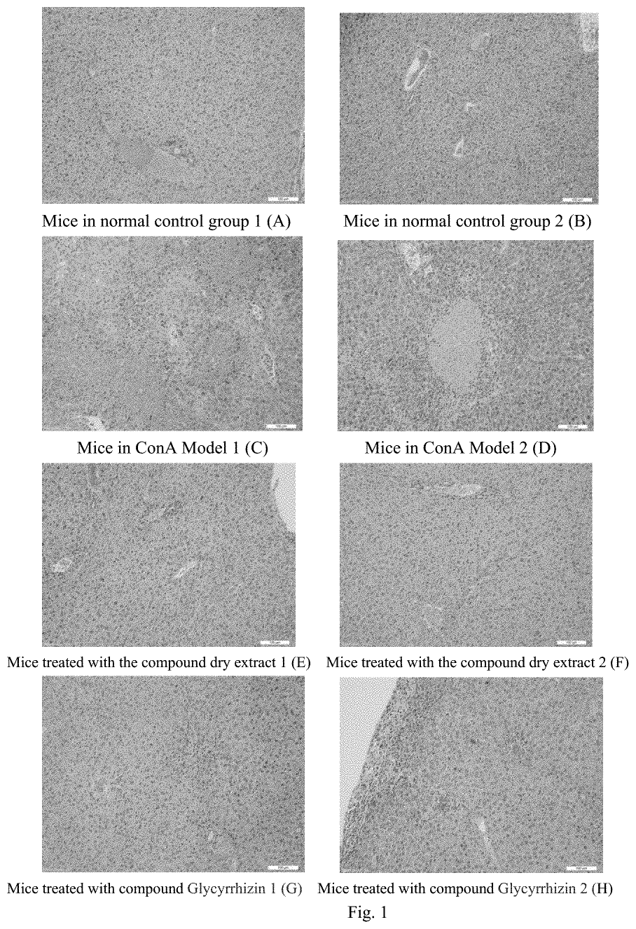

(3) Pathological analyses showed that: compared to the normal control group (A and B in FIG. 1), a plurality of hepatocyte necrosis areas were observed in the livers of the mice in the ConA-induced liver injury model group, and a large amount of inflammatory cell aggregated around these areas (C and D in FIG. 1) and fibrocyte proliferated. Compared to the model group, administrating with the compound dry extract to the mice could lead to apparent reduction or disappearance of the mouse hepatocyte necrosis areas, significant reduction of inflammatory cells, and meanwhile lead to the generation of binuclear hepatocytes and the increase of cell cleavages and proliferations (E-F in FIG. 1).

(4) The compound dry extract can reduce the increase of liver ALT and AST enzyme activities in ConA-induced mice (see Table 7).

TABLE 7
Effect of the compound dry extract on liver ALT and
AST enzyme activities in mice with liver injury

| | Dosage | | |
|---|---|---|---|
| Group | (g/kg) | ALT (U/L) | AST (U/L) |
| Normal control group | — | 12.0 ± 3.1 | 58.2 ± 19.2 |
| Model group | 0.01 | 37.2 ± 7.2*** | 87.1 ± 22.6* |
| Compound dry extract | 2.0 | 28.9 ± 7.2 | 57.9 ± 15.8 |
| group | | | |
| Compound | 0.05 | 59.1 ± 7.2^{##} | 47.0 ± 16.0^{##} |
| glycyrrhizin tablet | | | |
| group | | | |

Note:
as compared to the normal group, *P < 0.05, ***P < 0.001; as compared to the model group, ^{#}P < 0.05, ^{##}P < 0.01.

### Example 38: Evaluation on Activity of Promoting Hepatocyte Proliferation

1. Test Samples: The Samples Prepared According to the Methods in Example 21 and Example 26

2. Method

LO2 hepatocytes (a gift from professor Liqian of our institute) in logarithmic growth phase were dissociated and formulated into single cell suspension with 1% FBS RPMI-1640 solution, the single cell suspension was stained with Tryran blue solution, and total active cells were counted, and then inoculated into 96-well cell culture plate with cell density of 1×10⁴/well. The test samples were formulated with the culture solution and added into the culture plate so that the final concentrations were 0, 10, 50 and 250 μg·mL⁻¹, triplicate wells were set up for each concentration in parallel, and the plate was placed in the incubator and incubated for 72 hours. 20 μL of 0.5% MTT solution was added into each well of the 96-well plate containing the culture solution, the culture was conducted for another 4 h, and then 100 μL of 10% SDS was added into each well and cultured at 37° C. overnight. At last, enzyme-labelling meter was used to detect the OD value at 570 nm wavelength to calculate the cell proliferation rate.

3. Results

Effect of the compound dry extract of water decoction liquor and the polysaccharide fraction and alcohol precipitation supernatant fraction thereof on promoting hepatocyte proliferation were shown in Table 8.

TABLE 8
Effect of the compound dry extract and two fractions
thereof on proliferation of human LO2 hepatocyte.

| | Concentration | | Proliferation |
|---|---|---|---|
| Sample | (μg/mL) | OD_{570 nm} | Rate (%) |
| Solvent control | — | 0.700 ± 0.021 | — |
| Compound dry extract | 10 | 0.769 ± 0.022** | 9.8 |
| | 50 | 0.846 ± 0.008*** | 20.8 |
| | 250 | 1.232 ± 0.083*** | 76.0 |
| Polysaccharide | 10 | 0.806 ± 0.005*** | 15.2 |
| fraction | 50 | 0.961 ± 0.016*** | 37.3 |
| | 250 | 1.227 ± 0.064*** | 75.3 |
| Alcohol precipitation | 10 | 0.754 ± 0.021* | 7.6 |
| supernatant fraction | 50 | 0.900 ± 0.026*** | 28.5 |
| | 250 | 1.113 ± 0.031*** | 59.0 |

Note:
*P < 0.05, **P < 0.01, ***P < 0.001, n = 3, as compared to the solvent control group.

The results showed that the compound dry extract of water decoction liquor and two fractions thereof have apparent activity of promoting hepatocyte proliferation in a concentration ranging from 10 μg/ml to 250 μg/ml, and they exhibited good dose-effect relationship.

### Example 39: Evaluation on the Activity Against Hepatocyte Injury

1. Test Samples: The Compound Dry Extract Prepared According to the Method in Example 21 and the Compound Alcohol Precipitation Supernatant and Polysaccharide Prepared According to the Method in Example 26

2. Method

Human LO2 hepatocytes in logarithmic growth phase were dissociated and formulated into single cell suspension with 1% FBS RPMI-1640 solution, the single cell suspension was stained with Tryran blue solution, and total active cells were counted, and then inoculated into 96-well cell culture plate with cell density of 1×10⁴/well. The test samples were formulated with the culture solution and added into the culture plate so that the final concentrations were 0, 10, 50 and 250 μg·mL⁻¹, Con A was then added to each well with final concentration of 500 μg/mL, solvent control group was set up at the same time. Triplicate wells were set up for each concentration in parallel, and the plate was placed in the incubator and incubated for 72 hours. 20 μL of 0.5% MTT solution was added into each well of the 96-well plate containing the culture solution, the culture was conducted for another 4 h, and then 100 μL of 10% SDS was added into each well and cultured at 37° C. overnight. At last, enzyme-labelling meter was used to detect the OD value at 570 nm wavelength to calculate the cell inhibition rate.

3. Results

Compared to the solvent control group, 500 μg/mL ConA can result in 55.8% hepatocyte death, whereas by adding the compound dry extract, the compound polysaccharide fraction or the compound alcohol precipitation supernatant fraction, the hepatocyte death rate was remarkably reduced, and in a concentration-effect relationship. The dry extract and the polysaccharide can further promote the increase of the number of hepatocytes at the concentration of 250 μg/mL, and the specific data were shown in Table 9.

TABLE 9
Inhibition of the compound extract compound and two
fractions thereof against human LO2 hepatocyte injury

| | Concentration | | Death rate |
|---|---|---|---|
| Sample | (μg/mL) | OD_{570 nm} | (%) |
| Solvent control | — | 0.695 ± 0.011 | — |
| ConA | 500 | 0.307 ± 0.011*** | 55.8 |
| Compound dry extract | 10 | 0.353 ± 0.003^{##} | 49.2 |
| | 50 | 0.421 ± 0.019^{##} | 39.4 |
| | 250 | 0.716 ± 0.030^{###} | −3.1 |
| Polysaccharide | 10 | 0.418 ± 0.004^{###} | 39.9 |
| fraction | 50 | 0.598 ± 0.005^{###} | 13.9 |
| | 250 | 0.790 ± 0.009^{###} | −13.8 |
| Alcohol precipitation | 10 | 0.359 ± 0.015^{#} | 48.3 |
| supernatant fraction | 50 | 0.452 ± 0.004^{###} | 34.9 |
| | 250 | 0.695 ± 0.013^{###} | 0.0 |

Note:
as compared to the solvent control group, ***P < 0.001; as compared to the ConA group, ^{#}P < 0.05, ^{##}P < 0.01, ^{###}P < 0.001, n = 3.

### Example 40: Evaluation on the Activity of Promoting Hepatocyte Proliferation

1. Test Samples: The Samples Prepared According to the Methods in Example 22, Example 23, Example 24 and Example 25

2. Method

LO2 hepatocytes in logarithmic growth phase were dissociated and formulated into single cell suspension with 1% FBS RPMI-1640 solution, the single cell suspension was stained with Tryran blue solution, and total active cells were counted, and then inoculated into 96-well cell culture plate with cell density of 1×10⁴/well. The test samples were formulated with the culture solution and added into the culture plate so that the final concentrations were 0, 10, 50 and 250 μg·mL⁻¹, triplicate wells were set up for each concentration in parallel, and the plate was placed in the incubator and incubated for 72 hours. 20 μL of 0.5% MTT solution was added into each well of the 96-well plate containing the culture solution, the culture was conducted for another 4 h, and then 100 μL of 10% SDS was added into each well and cultured at 37° C. overnight. At last, enzyme-labelling meter was used to detect the OD value at 570 nm wavelength to calculate the cell proliferation rate.

3. Results

As seen from Table 10, the dry extract samples prepared according to the methods in examples 22-25 each can remarkably promote human hepatocyte proliferation and in good dose-effect relationship.

TABLE 10
Effect of the three compound extracts
on human LO2 hepatocyte proliferation

| | Concentration | | Proliferation |
|---|---|---|---|
| Group | (μg/mL) | OD_{(x±s)} | rate (%) |
| Solvent control | — | 0.607 ± 0.027 | |
| Sample of | 10 | 0.621 ± 0.004 | 2.3 |
| Example 22 | 50 | 0.704 ± 0.004** | 16.0 |
| | 250 | 1.150 ± 0.014*** | 89.5 |
| Sample of | 10 | 0.614 ± 0.015 | 1.2 |
| Example 23 | 50 | 0.708 ± 0.017* | 16.6 |
| | 250 | 0.822 ± 0.037** | 35.4 |
| Sample of | 10 | 0.615 ± 0.020 | 1.3 |
| Example 24 | 50 | 0.666 ± 0.040 | 9.7 |
| | 250 | 0.944 ± 0.018*** | 55.5 |
| Sample of | 10 | 0.645 ± 0.007 | 6.3 |
| Example 25 | 50 | 0.649 ± 0.012 | 6.9 |
| | 250 | 0.907 ± 0.033*** | 49.4 |

Note:
as compared to the solvent control group, *P < 0.05, **P < 0.01, ***P < 0.001, n = 3.

### Example 41: Evaluation on Expression of Hepatitis B Antigen

1. Test Substances: The Samples Prepared According to the Methods in Example 21 and Example 26

2. Method

HepG2.2.15 cells (a gift from Institute Pasteur of Shanghai Chinese of Academy of Sciences) in logarithmic growth phase were dissociated with pancreatin, DMEM culture solution (containing 10% FBS) was added to terminate the dissociation, and then transferred into a 15 ml centrifuge tube and centrifuged for 5 min at 1000 rpm; after removing the liquids, the resulting mixture was added with 1 ml of the DMEM solution (containing 10% FBS) for suspension, and counted. The cells were incubated in 96-well plate at 5.0×10³ cells/well, for each well, 100 μl DMEM medium (containing 10% FBS) was contained, and the plate was placed in 5% CO₂ incubator at 37° C. in saturated humidity for 24 hours or 48 hours. After the cells adhered to the walls, the original culture solution was discarded, the culture solutions of the test samples were respectively added in 100 μl/well (triplicate wells were set up in 100, 250 and 500 μg/mL), the supernatants of the cell cultures were respectively collected at 24 hours after drug intervention. The HBsAg and HBeAg levels in the cell culture supernatants were detected by using hepatitis B virus surface antigen (HBsAg) diagnostic kit (enzyme-linked immunosorbent assay) and hepatitis B virus e antigen (HBeAg) diagnostic kit (enzyme-linked immunosorbent assay), and the inhibition rate was calculated.

3. Results

The compound dry extract and the compound polysaccharide fraction had remarkable inhibitions on the secretion of hepatitis B virus surface antigens (HBsAg) and in good concentration-effect relationship; the compound dry extract and its two fractions namely the alcohol precipitation supernatant and polysaccharide fraction had remarkable inhibitions on the secretion of hepatitis B virus e antigens (HBeAg), but the compound dry extract and the compound polysaccharide fraction had more evident dose-effect relationship. The specific data were shown in Tables 11 and 12.

TABLE 11
Effect of the compound dry extract and two fractions thereof
on the expression of hepatitis B surface antigen (HBsAg)

| | Concentration | | Inhibition |
|---|---|---|---|
| Sample | μg/mL | OD_{570 nm} | ratio % |
| Solvent control group | | 3.093 ± 0.289 | |
| Compound dry extract | 500 | 1.761 ± 0.089 | 43.0 ± 2.9 |
| | 250 | 2.040 ± 0.111 | 34.0 ± 3.6 |
| | 100 | 2.580 ± 0.008 | 16.6 ± 0.2 |
| | 50 | 3.190 ± 0.155 | −3.1 ± 5.0 |
| Alcohol precipitation | 500 | 2.865 ± 0.112 | 7.4 ± 3.6 |
| supernatant fraction | 250 | 3.122 ± 0.062 | −0.9 ± 2.0 |
| | 100 | 3.127 ± 0.023 | −1.1 ± 0.7 |
| | 50 | 3.429 ± 0.060 | −10.9 ± 1.9 |
| Polysaccharide | 500 | 2.287 ± 0.128 | 36.1 ± 4.1 |
| fraction | 250 | 2.369 ± 0.252 | 23.4 ± 8.1 |
| | 100 | 2.669 ± 0.202 | 13.4 ± 6.3 |
| | 50 | 2.881 ± 0.148 | 6.8 ± 4.8 |

TABLE 12
Effect of the compound dry extract and two fractions thereof
on expression of hepatitis B core antigen (HBeAg)

| | Concentration | | Inhibition |
|---|---|---|---|
| Sample | μg/mL | OD_{570 nm} | ratio % |
| Solvent control group | | 0.791 ± 0.100 | |
| Compound dry extract | 500 | 0.438 ± 0.033 | 44.7 ± 4.27 |
| | 250 | 0.613 ± 0.098 | 22.5 ± 12.47 |
| | 100 | 0.666 ± 0.023 | 15.8 ± 2.9 |
| | 50 | 0.736 ± 0.039 | 7.0 ± 4.9 |
| Compound alcohol | 500 | 0.565 ± 0.020 | 28.6 ± 2.5 |
| precipitation | 250 | 0.647 ± 0.057 | 18.1 ± 7.2 |
| supernatant | 100 | 0.618 ± 0.160 | 21.8 ± 20.2 |
| fraction | 50 | 0.818 ± 0.045 | −3.4 ± 5.7 |
| Compound | 500 | 0.503 ± 0.040 | 36.4 ± 5.0 |
| polysaccharide | 250 | 0.537 ± 0.025 | 32.1 ± 3.2 |
| fraction | 100 | 0.610 ± 0.061 | 22.9 ± 7.7 |
| | 50 | 0.792 ± 0.062 | 7.9 ± 0.1 |

### Example 42: Evaluation on Immunity Activity

1. Test Samples: The Samples Prepared According to the Methods in Example 21 and Example 26

2. Method

(1) Method for Evaluating the Activity of Enhancing Proliferation of Mouse Splenocytes

Balc/C mice were bled from eyeballs and sacrificed by cervical dislocation method. The spleen was taken out under aseptic condition to prepare splenocyte suspension, and the cell survival rate of >95% was determined by trypan blue staining method, and cell counting was performed to adjust cell density to 5×10⁶/mL. The counted splenocyte suspension was added into 96-well cell culture plate in 100 μL per well, and solvent control wells were only added with equivalent volume of RPMI1640 culture solution as background. Each well was further added with 100 μL of the test sample (with the final concentrations of 10, 50 and 250 μg/mL, respectively), ConA (with the final concentration of 2 μg/mL) and LPS (with the final concentration of 15 μg/mL), triplicate wells were set up for each group. The culture plate was placed in an incubator with 5% CO₂, and incubated at 37° C. for 72 h, then added with 20 μL of MTT 5 μg/mL, the incubation was continued for 4 h. After the supernatant was discarded, each well was added with 150 μL of DMSO, and enzyme-labelled meter was used to detect the OD value at A_{570 nm} to calculate the cell proliferation rate. The results were shown in Table 13.

TABLE 13
Effect of the compound dry extract of water decoction and
fractions thereof on mouse splenocyte proliferations

| | Dosage | OD_{570 nm} | | |
|---|---|---|---|---|
| Group | (μg/mL) | RPMI1640 | ConA | LPS |
| Solvent control | — | 0.560 ± 0.013 | 0.56 ± 0.031 | 0.534 ± 0.035 |
| ConA | 2 | | 0.859 ± 0.003*** | |
| LPS | 15 | | | 0.719 ± 0.008** |
| Compound dry extract | 10 | 0.561 ± 0.011 | 0.821 ± 0.012 | 0.751 ± 0.012^{#} |
| | 50 | 0.564 ± 0.017 | 0.845 ± 0.041 | 0.782 ± 0.023^{#} |
| | 250 | 0.569 ± 0.021 | 0.702 ± 0.107 | 0.784 ± 0.009^{#} |
| Polysaccharide | 10 | 0.556 ± 0.011 | 0.906 ± 0.102 | 0.691 ± 0.117 |
| fraction | 50 | 0.597 ± 0.034 | 0.827 ± 0.025 | 0.785 ± 0.005^{###} |
| | 250 | 0.772 ± 0.007*** | 0.910 ± 0.038 | 0.884 ± 0.025^{###} |
| Alcohol precipitation | 10 | 0.574 ± 0.014 | 0.851 ± 0.028 | 0.777 ± 0.018^{#} |
| supernatant fraction | 50 | 0.589 ± 0.018 | 0.883 ± 0.041 | 0.749 ± 0.023 |
| | 250 | 0.608 ± 0.006** | 0.751 ± 0.016^{###} | 0.712 ± 0.005 |

Note:
as compared to the solvent control group, **P < 0.01, ***P < 0.001; as compared to ConA or LPS group, ^{#}P < 0.05, ^{##}P < 0.01, ^{###}P < 0.001, n = 3.

The results showed that the polysaccharide fraction and alcohol precipitation supernatant fraction of the compound dry extract would remarkably promote the proliferation activity of mouse splenocytes at 250 m/mL, the alcohol precipitation supernatant fraction promoted ConA-stimulated T cell proliferation synergistically, and the polysaccharide fraction promoted LPS-simulated B cell proliferation synergistically.

(2) Method for Measuring Cytokines IFN-γ and TNF-α

Spleens were aseptically taken out after Balb/C mice were sacrificed to prepare splenocyte suspension (5×10⁶/mL). The splenocyte suspension was added to 24-well cell culture plate at 500 μL per well. Then 500 μL of the test samples (the final concentrations of 10, 50 and 250 μg/mL, respectively) were added, and ConA (4 μg/mL) and solvent control wells were additionally set up, triplicate wells were set up for each group. Then, the plate was placed in 5% CO₂ incubator at 37° C. for incubation for 72 hours, and then the cell culture solution was taken out and centrifuged for 10 min (600×g). The supernatant was collected, and the content of IFN-γ in the splenocyte culture supernatant was measured according to the specification of the ELISA kit. The test results were shown in Table 14.

Peritoneal macrophages were extracted aseptically after Balb/C mice were sacrificed to prepare cell suspension, which was adjusted with DMEM culture solution containing FBS to reach cell concentration of 2.5×10⁵/mL. 500 μL of the peritoneal macrophage suspension was added to 24-well cell culture plate. Then 500 μL of the test samples (the final concentrations of 10, 50 and 250 μg/mL, respectively) were added, and LPS (15 μg/mL) and solvent control wells were additionally set up, triplicate wells were set up for each group. Then, the plate was placed in 5% CO₂ incubator at 37° C. for incubation for 48 hours, and then the supernatant of the peritoneal macrophage culture was collected, and the content of TNF-α in the supernatant was measured by using ELISA kit. The test results were shown in Table 15.

TABLE 14
Effect of the compound dry extract of water decoction and fractions
thereof on the secretion of IFN-γ of mouse spleen lymphocytes

| | Dose | IFN-γ | |
|---|---|---|---|
| Group | (μg/ml) | OD_{450 nm} | pg/mL |
| Solvent control | — | 0.138 ± 0.006 | 1.48 ± 1.06 |
| ConA | 2 | 5.198 ± 0.135*** | 841.91 ± 22.49*** |
| Compound dry | 10 | 0.138 ± 0000 | 1.49 ± 0.04 |
| extract | 50 | 0.132 ± 0.003 | 2.47 ± 0.55 |
| | 250 | 0.134 ± 0.003 | 2.09 ± 0.46 |
| Polysaccharide | 10 | 0.140 ± 0.010 | 1.09 ± 1.63 |
| fraction | 50 | 0.196 ± 0.019 | 8.17 ± 3.24 |
| | 250 | 0.268 ± 0.036* | 20.17 ± 6.04* |
| Alcohol | 10 | 0.138 ± 0.005 | 1.48 ± 0.85 |
| precipitation | 50 | 0.140 ± 0.004 | 1.14 ± 0.61 |
| supernatant | 250 | 0.138 ± 0.003 | 1.57 ± 0.51 |
| fraction | | | |

Note:
as compared to the solvent control group, *P < 0.05, ***P < 0.001, n = 3.

TABLE 15
Effect of the compound dry extract of water decoction and fractions
thereof on the secretion of TNF-α of mouse peritoneal macrophages

| | |
|---|---|
| Dose | TNF-α |

| Group | (μg/ml) | OD_{450 nm} | pg/mL |
|---|---|---|---|
| Solvent control | — | 0.230 ± 0.012 | 37.25 ± 6.01 |
| LPS | 15 | 0.281 ± 0.002* | 62.75 ± 1.06** |
| Compound dry | 10 | 0.274 ± 0.008 | 59.50 ± 4.24 |
| extract | 50 | 0.294 ± 0.014* | 69.50 ± 7.07* |
| | 250 | 0.318 ± 0.027* | 81.50 ± 13.43* |
| Polysaccharide | 10 | 0.283 ± 0.010* | 64.00 ± 4.95* |
| fraction | 50 | 0.285 ± 0.025 | 64.75 ± 12.37 |
| | 250 | 0.204 ± 0.001 | 24.25 ± 0.35 |
| Alcohol | 10 | 0.272 ± 0.011 | 58.25 ± 5.30 |
| precipitation | 50 | 0.297 ± 0.016* | 70.75 ± 8.13* |
| supernatant | 250 | 0.331 ± 0.021* | 88.00 ± 10.61* |
| fraction | | | |

Note:
as compared to the solvent control group, *P < 0.05, **P < 0.01, ***P < 0.001, n = 3.

The results showed that the compound polysaccharide can promote the mouse splenocytes to secrete IFN-γ, and the compound dry extract and its two fractions are all able to enhance the secretion of TNF-α of mouse peritoneal macrophages.

### Example 43: Evaluation on the Activity of Promoting Splenocyte Proliferation

1. Test Samples: The Samples Prepared According to the Methods in Examples 22-25

2. Method

Balc/C mice were bled from eyeballs and sacrificed by cervical dislocation method. The spleen was taken out under aseptic condition to prepare splenocyte suspension, and the cell survival rate of >95% was determined by trypan blue staining method, and cell counting was performed to adjust cell density to 5×10⁶/mL. The counted splenocyte suspension was added into 96-well cell culture plate in 100 μL per well, and solvent control wells were only added with equivalent volume of RPMI1640 culture solution as background. Each well was further added with 100 μL of the test sample (with the final concentrations of 10, 50 and 250 m/mL, respectively), ConA (with the final concentration of 2 μg/mL) and LPS (with the final concentration of 15 μg/mL), triplicate wells were set up for each group. The culture plate was placed in an incubator with 5% CO₂, and incubated at 37° C. for 72 h, then added with 20 μL of MTT 5 μg/mL, the incubation was continued for 4 h. After the supernatant was discarded, each well was added with 150 μL of DMSO, and enzyme-labelled meter was used to detect the OD value at A_{570 nm}.

3. Results

The data showed that the dry extracts prepared according to the methods in examples 22-25 had evident activities of promoting mouse splenocyte proliferations at 250 μg/ml, and the extracts at this concentration promoted ConA-induced T cell proliferation synergistically, the extract having anti-hepatic fibrosis activity also can promote the LPS-induced B cell proliferations synergistically (see Table 16).

TABLE 16
Effect of the four effective compound dry extracts on mouse splenocyte proliferation

| | Dose | OD_{570 nm} | | |
|---|---|---|---|---|
| Group | (μg/ml) | RPMI1640 | ConA | LPS |
| Solvent control | — | 0.578 ± 0.049 | 0.538 ± 0.030 | 0.626 ± 0.017 |
| ConA | 2 | | 0.872 ± 0.017*** | |
| LPS | 15 | | | 0.820 ± 0.026*** |
| extract with anti- | 10 | 0.601 ± 0.018 | 0.889 ± 0.010 | 0.850 ± 0.071 |
| nonalcoholic liver | 50 | 0.611 ± 0.041 | 0.906 ± 0.070 | 0.845 ± 0.059 |
| activity | 250 | 0.661 ± 0.022* | 0.828 ± 0.021^{#} | 0.880 ± 0.005 |
| extract with anti- | 10 | 0.595 ± 0.035 | 0.913 ± 0.033 | 0.866 ± 0.062 |
| alcoholic liver | 50 | 0.598 ± 0.015 | 0.893 ± 0.049 | 0.860 ± 0.006 |
| activity | 250 | 0.637 ± 0.027* | 0.823 ± 0.022^{#} | 0.771 ± 0.030 |
| extract with anti- | 10 | 0.613 ± 0.038 | 0.913 ± 0.026 | 0.882 ± 0.089 |
| fibrosis activity | 50 | 0.642 ± 0.040 | 0.896 ± 0.059 | 0.911 ± 0.051^{#} |
| | 250 | 0.679 ± 0.003* | 0.989 ± 0.024* | 0.959 ± 0.046^{#} |
| extract with anti- | 10 | 0.543 ± 0.023 | 0.951 ± 0.053 | 0.715 ± 0.014 |
| liver cancer | 50 | 0.614 ± 0.037 | 0.906 ± 0.043 | 0.782 ± 0.029 |
| activity | 250 | 0.675 ± 0.025* | 0.950 ± 0.112 | 0.830 ± 0.01 |

Note:
as compared to the solvent control group, *P < 0.05, ***P < 0.001; as compared to ConA or LPS group, ^{#}P < 0.05, n = 3.

Though specific embodiments of the invention have been described in detail, according to all teachings that have been disclosed, those skilled in the art could make various modifications and alternatives to those details in the technical solution of the invention, and these changes each are in the protection scope of the invention. The whole scope of the invention is given by the attached claims and any equivalents thereof.

1. A traditional Chinese medicine composition, comprising the following components: Morinda officinalis How, Ganoderma lucidum (Leyss. Ex Fr.) Karst., Astragalus membranaceus (Fisch) Bge. Var. monghlicus (Bge) Hsiao, Ranunculus ternatus Thunb., Sophora flavescens Alt. and Salvia miltiorrhiza Bge.

2. The traditional Chinese medicine composition as claimed in claim 1, wherein weight ratios of the components are: 15-35 parts of Morinda officinalis How, 10-20 parts of Ganoderma lucidum (Leyss. Ex Fr.) Karst., 25-35 parts of Astragalus membranaceus (Fisch) Bge. Var. monghlicus (Bge) Hsiao, 10-20 parts of Ranunculus ternatus Thunb., 10-20 parts of Sophora flavescens Alt., and 10-20 parts of Salvia miltiorrhiza Bge.;
preferably, weight ratios of the components are: 30 parts of Morinda officinalis How, 15 parts of Ganoderma lucidum (Leyss. Ex Fr.) Karst., 30 parts of Astragalus membranaceus (Fisch) Bge. Var. monghlicus (Bge) Hsiao, 15 parts of Ranunculus ternatus Thunb., 15 parts of Sophora flavescens Alt. and 15 parts of Salvia miltiorrhiza Bge.

3. The traditional Chinese medicine composition as claimed in claim 1 or 2, further comprising one or more (e.g., 2-10, e.g., 2-6, e.g., 2, 3, 4, 5, or 6) of the following components: Epimedium brevicornu Maxim., Rehmannia glutinosa Libosch., Atractylodes macrocephala Koidz., Citrus reticulata Blanco, Sedum sarmentosum Bunge, Pueraria lobata (Willd.) Ohwi, Hovenia acerba Lindl., Flower of Pueraria lobata (Willd.) Ohwi, Lycopus lucidus Turcz. var. hirtus Regel, Curcuma wenyujin Y. H. Chen et C. Ling, Stir-baked Trionyx sinensis Wiegmann, Dendrobium nobile Lindl., Cremastra appendiculata Makino, Abrus cantoniensis Hance, Curcuma phaeocaulis Val., and Scutellaria barbata D. Don;
preferably, the traditional Chinese medicine composition further comprises the following components: Epimedium brevicornu Maxim., Rehmannia glutinosa Libosch., Atractylodes macrocephala Koidz., and Citrus reticulata Blanco; further preferably, the weight ratios of the components are: 15-35 parts of Morinda officinalis How, 10-20 parts of Ganoderma lucidum (Leyss. Ex Fr.) Karst., 25-35 parts of Astragalus membranaceus (Fisch) Bge. Var. monghlicus (Bge) Hsiao, 10-20 parts of Ranunculus ternatus Thunb., 10-20 parts of Sophora flavescens Alt., 10-20 parts of Salvia miltiorrhiza Bge., 10-30 parts of Epimedium brevicornu Maxim., 5-15 parts of Rehmannia glutinosa Libosch., 10-30 parts of Atractylodes macrocephala Koidz., and 5-10 parts of Citrus reticulata Blanco; further preferably, the weight ratios of the components are: 30 parts of Morinda officinalis How, 15 parts of Ganoderma lucidum (Leyss. Ex Fr.) Karst., 30 parts of Astragalus membranaceus (Fisch) Bge. Var. monghlicus (Bge) Hsiao, 15 parts of Ranunculus ternatus Thunb., 15 parts of Sophora flavescens Alt., 15 parts of Salvia miltiorrhiza Bge., 15 parts of Epimedium brevicornu Maxim., 10 parts of Rehmannia glutinosa Libosch., 15-30 parts of Atractylodes macrocephala Koidz. and 9-10 parts of Citrus reticulata Blanco;
preferably, the traditional Chinese medicine composition further comprises the following components: Sedum sarmentosum Bunge and Pueraria lobata (Willd.) Ohwi; further preferably, the weight ratios of the components are: 15-35 parts of Morinda officinalis How, 10-20 parts of Ganoderma lucidum (Leyss. Ex Fr.) Karst., 25-35 parts of Astragalus membranaceus (Fisch) Bge. Var. monghlicus (Bge) Hsiao, 10-20 parts of Ranunculus ternatus Thunb., 10-20 parts of Sophora flavescens Alt., 10-20 parts of Salvia miltiorrhiza Bge., 15-30 parts of Sedum sarmentosum Bunge, and 15-30 parts of Pueraria lobata (Willd.) Ohwi; further preferably, the weight ratios of the components are: 30 parts of Morinda officinalis How, 15 parts of Ganoderma lucidum (Leyss. Ex Fr.) Karst., 30 parts of Astragalus membranaceus (Fisch) Bge. Var. monghlicus (Bge) Hsiao, 15 parts of Ranunculus ternatus Thunb., 10 parts of Sophora flavescens Alt., 15 parts of Salvia miltiorrhiza Bge., 30 parts of Sedum sarmentosum Bunge and 30 parts of Pueraria lobata (Willd.) Ohwi;
preferably, the traditional Chinese medicine composition further comprises the following components: Sedum sarmentosum Bunge, Pueraria lobata (Willd.) Ohwi, Hovenia acerba Lindl. and Flower of Pueraria lobata (Willd.) Ohwi; further preferably, the weight ratios of the components are: 15-35 parts of Morinda officinalis How, 10-20 parts of Ganoderma lucidum (Leyss. Ex Fr.) Karst., 25-35 parts of Astragalus membranaceus (Fisch) Bge. Var. monghlicus (Bge) Hsiao, 10-20 parts of Ranunculus ternatus Thunb., 10-20 parts of Sophora flavescens Alt., 10-20 parts of Salvia miltiorrhiza Bge., 15-30 parts of Sedum sarmentosum Bunge, 15-30 parts of Pueraria lobata (Willd.) Ohwi, 15-30 parts of Hovenia acerba Lindl., and 10-20 parts of Flower of Pueraria lobata (Willd.) Ohwi; further preferably, the weight ratios of the components are: 30 parts of Morinda officinalis How, 15 parts of Ganoderma lucidum (Leyss. Ex Fr.) Karst., 30 parts of Astragalus membranaceus (Fisch) Bge. Var. monghlicus (Bge) Hsiao, 15 parts of Ranunculus ternatus Thunb., 10 parts of Sophora flavescens Alt. 15 parts of Salvia miltiorrhiza Bge., 30 parts of Sedum sarmentosum Bunge, 30 parts of Pueraria lobata (Willd.) Ohwi, 30 parts of Hovenia acerba Lindl., and 15 parts of Flower of Pueraria lobata (Willd.) Ohwi;
preferably, the traditional Chinese medicine composition further comprises the following components: Lycopus lucidus Turcz. var. hirtus Regel, Curcuma wenyujin Y. H. Chen et C. Ling, Stir-baked Trionyx sinensis Wiegmann and Dendrobium nobile Lindl.; further preferably, the weight ratios of the components are: 15-35 parts of Morinda officinalis How, 10-20 parts of Ganoderma lucidum (Leyss. Ex Fr.) Karst., 25-35 parts of Astragalus membranaceus (Fisch) Bge. Var. monghlicus (Bge) Hsiao, 10-20 parts of Ranunculus ternatus Thunb., 10-20 parts of Sophora flavescens Alt., 10-20 parts of Salvia miltiorrhiza Bge., 10-20 parts of Lycopus lucidus Turcz. var. hirtus Regel, 10-20 parts of Curcuma wenyujin Y. H. Chen et C. Ling, 10-20 parts of Stir-baked Trionyx sinensis Wiegmann and 10-20 parts of Dendrobium nobile Lindl.; further preferably, the weight ratios of the components are: 30 parts of Morinda officinalis How, 15 parts of Ganoderma lucidum (Leyss. Ex Fr.) Karst., 30 parts of Astragalus membranaceus (Fisch) Bge. Var. monghlicus (Bge) Hsiao, 15 parts of Ranunculus ternatus Thunb., 10 parts of Sophora flavescens Alt., 15 parts of Salvia miltiorrhiza Bge., 15 parts of Lycopus lucidus Turcz. var. hirtus Regel, 15 parts of Curcuma wenyujin Y. H. Chen et C. Ling, 15 parts of Stir-baked Trionyx sinensis Wiegmann and 15 parts of Dendrobium nobile Lindl.;
preferably, the traditional Chinese medicine composition further comprises the following components: Cremastra appendiculata Makino, Abrus cantoniensis Hance, Curcuma phaeocaulis Val., Atractylodes macrocephala Koidz. and Scutellaria barbata D. Don; further preferably, the weight ratios of the components are: 15-35 parts of Morinda officinalis How, 10-20 parts of Ganoderma lucidum (Leyss. Ex Fr.) Karst., 25-35 parts of Astragalus membranaceus (Fisch) Bge. Var. monghlicus (Bge) Hsiao, 10-20 parts of Ranunculus ternatus Thunb., 10-20 parts of Sophora flavescens Alt., 10-20 parts of Salvia miltiorrhiza Bge., 5-10 parts of Cremastra appendiculata Makino, 15-30 parts of Abrus cantoniensis Hance, 5-10 parts of Curcuma phaeocaulis Val., 20-30 parts of Atractylodes macrocephala Koidz. and 10-20 parts of Scutellaria barbata D. Don; further preferably, the weight ratios of the components are: 30 parts of Morinda officinalis How, 15 parts of Ganoderma lucidum (Leyss. Ex Fr.) Karst., 30 parts of Astragalus membranaceus (Fisch) Bge. Var. monghlicus (Bge) Hsiao, 15 parts of Ranunculus ternatus Thunb., 10 parts of Sophora flavescens Alt., 15 parts of Salvia miltiorrhiza Bge., 6 parts of Cremastra appendiculata Makino, 15 parts of Abrus cantoniensis Hance, 6 parts of Curcuma phaeocaulis Val., 15 parts of Atractylodes macrocephala Koidz. and 15 parts of Scutellaria barbata D. Don.

4. A traditional Chinese medicine extract A, which is prepared through the following method:
mixing the components of the traditional Chinese medicine composition as defined in any of claims 1 to 3, adding water and decocting to give a water decoction liquor; preferably, the volume of the water added is 5 to 20 times (in L: Kg) (e.g., 5, 10, 15, or 20 times) relative to the weight of the components;
concentrating the water decoction liquor to give a concentrated liquor, namely the traditional Chinese medicine extract A;
preferably, prior to adding water and decocting, further comprising a step of comminuting the components; further preferably, after the comminuting, further comprising a step of screening through a sieve (e.g., a 20-mesh sieve);
preferably, prior to concentrating, further comprising steps of filtering and/or centrifuging the water decoction liquor;
preferably, further comprising a step of drying (e.g., freeze drying) the concentrated liquor.

5. A traditional Chinese medicine extract B, which is prepared through the following method:
adding ethanol to the concentrated liquor as defined in claim 4 and letting it on stand for 12-72 h (such as 12 h, 24 h, 36 h, 48 h or 72 h) for precipitation; preferably, the volume of the ethanol added is 1 to 10 times (e.g., 2, 3, 5, 8, or 10 times) relative to the volume of the concentrated liquor;
collecting a supernatant and concentrating it to provide the traditional Chinese medicine extract B;
preferably, after the precipitation and prior to collecting the supernatant, further comprising a step of centrifuging;
preferably, after the concentrating, further comprising a step of drying.

6. A traditional Chinese medicine extract C, which is prepared through the following method:
adding ethanol to the concentrated liquor as defined in claim 4 and letting it on stand for 12-72 h (such as 12 h, 24 h, 36 h, 48 h or 72 h) for precipitation; preferably, the volume of the ethanol added is 1 to 10 times (e.g., 2, 3, 5, 8, or 10 times) relative to the volume of the concentrated liquor;
collecting the precipitate and dissolving it in water in a manner of low intensity and high frequency (the total volume of the water added is 1-3 times (in L: kg) relative to the weight of the medicine materials), intercepting the fraction with molecular weight >1000 Da by dialysis, and concentrating to provide the traditional Chinese medicine extract C;
preferably, after the concentrating, further comprising a step of drying.

7. A pharmaceutical composition, comprising the traditional Chinese medicine composition as claimed in any one of claims 1 to 3 or the traditional Chinese medicine extract as claimed in any one of claims 4 to 6, and optionally one or more pharmaceutically acceptable carriers or excipients;
preferably, the pharmaceutical composition is in the form of decoctions, oral liquids, medical teas, medical wines, granules, tablets, capsules, powder, pills, or extracts.

8. An adjuvant or immunomodulator, comprising the traditional Chinese medicine composition as claimed in any one of claims 1 to 3, the traditional Chinese medicine extract as claimed in any one of claims 4 to 6 or the pharmaceutical composition as claimed in claim 7.

9. A pharmaceutical combination, comprising the traditional Chinese composition as claimed in any one of claims 1 to 3, the traditional Chinese medicine extract as claimed in any one of claims 4 to 6 or the pharmaceutical composition as claimed in claim 7, and one or more antiviral drugs; preferably, the antiviral drug is a nucleoside antiviral drug; preferably, the antiviral drug is selected from the group consisting of Entecavir, Tenofovir, Telbivudine, Adefovir, and Lamivudine, preferably Entecavir.

10. Use of the traditional Chinese medicine composition as claimed in any one of claims 1 to 3, the traditional Chinese medicine extract as claimed in any one of claims 4 to 6, the pharmaceutical composition as claimed in claim 7 or the pharmaceutical combination as claimed in claim 9 in the manufacture of a medicament for the treatment of liver injury or a liver disease;
preferably, the liver disease is selected from chronic hepatitis (e.g. chronic hepatitis B), nonalcoholic steatohepatitis, alcoholic steatohepatitis, hepatic fibrosis, compensated cirrhosis, liver cancer (especially primary liver cancer caused by chronic hepatitis B);
preferably, the liver injury is the liver injury caused by drug or toxicant.

11. The traditional Chinese medicine composition as claimed in any one of claims 1 to 3, the traditional Chinese medicine extract as claimed in any one of claims 4 to 6, the pharmaceutical composition as claimed in claim 7 or the pharmaceutical combination as claimed in claim 9, for use in the treatment of liver injury or a liver disease;
preferably, the liver disease is selected from chronic hepatitis (e.g. chronic hepatitis B), nonalcoholic steatohepatitis, alcoholic steatohepatitis, hepatic fibrosis, compensated cirrhosis, liver cancer (especially primary liver cancer caused by chronic hepatitis B);
preferably, the liver injury is the liver injury caused by drug or toxicant.

12. A method for treating liver injury or a liver disease, comprising the step of administering an effective amount of the traditional Chinese medicine composition as claimed in any one of claims 1 to 3, the traditional Chinese medicine extract as claimed in any one of claims 4-6, the pharmaceutical composition as claimed in claim 7 or the pharmaceutical combination as claimed in claim 9 to an individual in need of such treatment;
preferably, the liver disease is selected from chronic hepatitis (e.g. chronic hepatitis B), nonalcoholic steatohepatitis, alcoholic steatohepatitis, hepatic fibrosis, compensated cirrhosis, liver cancer (especially primary liver cancer caused by chronic hepatitis B);
preferably, the liver injury is the liver injury caused by drug or toxicant.

13. Use of the traditional Chinese medicine composition as claimed in any one of claims 1 to 3, the traditional Chinese medicine extract as claimed in any one of claims 4 to 6, or the pharmaceutical composition as claimed in claim 7 as an adjuvant or immunomodulator.

14. Use of the traditional Chinese medicine composition as claimed in any one of claims 1 to 3, the traditional Chinese medicine extract as claimed in any one of claims 4 to 6, the pharmaceutical composition as claimed in claim 7, the adjuvant or immunomodulator as claimed in claim 8, or the combination administration as claimed in claim 9 for modulating, balancing or restoring immune functions of an individual.

15. A method of modulating, balancing or restoring immune functions, comprising the step of administering an effective amount of the traditional Chinese medicine composition as claimed in any one of claims 1 to 3, the traditional Chinese medicine extract as claimed in any one of claims 4 to 6, the pharmaceutical composition as claimed in claim 7, the adjuvant or immunomodulator as claimed in claim 8 or the pharmaceutical combination as claimed in claim 9 to an individual in need of such treatment.
